SEQUENCE LISTING (1 sequence)

SEQ ID NO: 1 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

(19) United States
(12) Patent Application Publication
(10) Pub. No.: US 2017/0335402 A1
(43) Pub. Date: Nov. 23, 2017

(54) DOT1L INHIBITORS AND USES THEREOF

(71) Applicant: Baylor College of Medicine, Houston, TX (US)

(72) Inventors: Rachel Rau, Houston, TX (US); Margaret A. Goodell, Houston, TX (US)

(21) Appl. No.: 15/502,623

(22) PCT Filed: Aug. 8, 2015
(86) PCT No.: PCT/US2015/044394

§ 371 Date: Feb. 8, 2017

Related U.S. Application Data
(60) Provisional application No. 62/035,373, filed on Aug. 8, 2014.

(51) Int. Cl. C12Q 1/68; A61K 31/519; A61K 31/52

(52) U.S. Cl. CPC C12Q 1/6886; A61K 31/52; A61K 31/519; C12Q 2600/154; C12Q 2600/156; C12Q 2600/106

(57) ABSTRACT

The present disclosure relates to methods of treating AML, associated with DNMT3A mutations by administering one or more DOT1L inhibitors or related pharmaceutical compositions to subjects in need thereof.

### RELATED APPLICATIONS

This application claims the benefit under 35 U.S.C. §119(e) of U.S. provisional application U.S. Ser. No. 62/035,373, filed Aug. 8, 2014, and entitled “DOT1L Inhibitors and Uses Thereof”, the entire content of which is incorporated herein by reference.

### FEDERALLY SPONSORED RESEARCH

This invention was made with support from government grant number NCI/NIH K12 CA090433-11 awarded by the National Institutes of Health and grant number DK092883-01 awarded by the National Institutes of Health. The government has certain rights in the invention.

### BACKGROUND

The genetic analysis of samples from patients with acute myeloid leukemia (AML) has identified a range of somatic mutations, including certain mutations in the DNA (cytosine-5)-methyltransferase 3A (DNMT3A) gene. DNMT3A is a DNA methyltransferase that catalyzes methylation of CpG sequences in DNA. Mutations of the DNA methyltransferase DNMT3A are often associated with poor prognosis in AML patients.

### SUMMARY

Aspects of the disclosure relate to methods and compositions for treating acute myeloid leukemia (AML) and other haem malignancies associated with mutations in DNA methyltransferase 3A (DNMT3A). Aspects of the disclosure are based, at least in part, on the determination that AML associated with one or more mutations in DNMT3A is responsive to the inhibition of DOT1L activity. Accordingly, in some embodiments, a subject having AML associated with one or more mutations in DNMT3A can be treated with one or more DOT1L inhibitor compounds as described herein. In some embodiments, a subject diagnosed with AML and having expanded methylation canyons associated with DNMT3A mutation can be treated with one or more DOT1L inhibitor compounds as described herein. In some embodiments, a subject having expanded methylation canyons associated with DNMT3A comprising HOX gene clusters can be treated with one or more DOT1L inhibitor compounds as described herein.

Accordingly, aspects of the disclosure provide methods and compositions for the treatment of AML. In some embodiments, aspects of the disclosure are useful to identify AML patients that are responsive to treatment with one or more DOT1L inhibitor compounds. In some embodiments, a subject having one or more clinical symptoms, gene expression markers, and/or other indicia of AML associated with mutation(s) in DNMT3A is identified as a candidate for treatment with a DOT1L inhibitor compound (e.g., as a subject in need of treatment with a DOT1L inhibitor compound). In some embodiments, the subject is treated with one or more DOT1L inhibitor compounds as described herein.

In some embodiments, a subject at risk of developing AML associated with mutation(s) in DNMT3A can be treated with one or more DOT1L inhibitor compounds to prevent or slow the progression of the disease.

Non-limiting examples of DOT1L inhibitor compounds include a compound of formula:

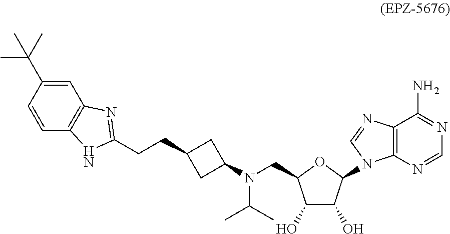 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, prodrug, metabolite, polymorph, solvate, or stereoisomer thereof, and a compound of formula:

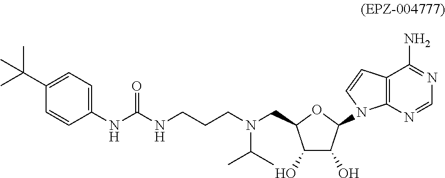 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, prodrug, metabolite, polymorph, solvate, or stereoisomer thereof.

In some embodiments, a DOT1L inhibitor is a compound of formula:

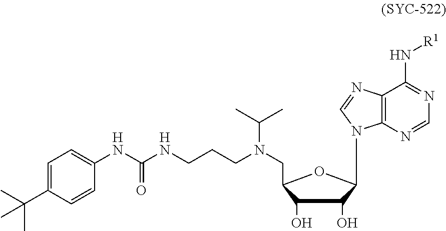

wherein R1 is a H, or a pharmaceutically acceptable salt, prodrug, metabolite, polymorph, solvate or stereoisomer thereof. However, it should be appreciated that other DOT1L inhibitors as described herein can be used.

Accordingly, the present disclosure provides methods and compositions for treating, preventing, and/or alleviating one or more symptoms of certain AMLs by administering to a subject in need thereof a therapeutically effective amount of a DOT1L inhibitor.

In some embodiments, a subject having AML associated with one or more mutations in DNMT3A can be identified by assaying a subject having AML (e.g., a biological sample obtained from the subject having AML) to determine whether one or more markers (e.g., genetic markers) of DNMT3A mutation(s) are present. In some embodiments, the presence of one or more dominant negative mutations in at least one allele of DNMT3A are detected in the subject (e.g., in a biological sample obtained from the subject). In some embodiments, the presence of one or more loss of function mutations (e.g., deletions or other loss of function mutations) in both alleles of DNMT3A are detected in the subject (e.g., in a biological sample obtained from the subject).

In adult AML, 20-25% of patients have DNMT3A mutations. Approximately 60% of these mutations are predicted to affect the R882 position that lies within the methyltransferase domain. Mutations affecting the R882 position result in a dominant negative loss of function (Russler-Germaine 2013, Kim 2013) and are almost exclusively heterozygous. The remainder of DNMT3A mutations are predicted to affect other amino acids (non-R882 mutations). Approximately 5-10% of patients with non-R882 mutations have either homozygous mutations or two different DNMT3A mutations. Accordingly, in some aspects, methods and compositions described herein can be used to treat subjects having AML associated with one or two DNMT3A mutations.

Hereditary mutations of DNMT3A have only recently been described in patients with an overgrowth syndrome with intellectual disability (Tatton-Brown Nat Genet. 2014). In some aspects, methods and compositions described herein can be used to treat subjects having AML associated with hereditary DNMT3A mutations.

In some aspects, a subject having AML associated with a DNMT3A mutation has one or more expanded methylation canyons (e.g., the length of one or more hypomethylated DNA regions is expanded). In some embodiments, an expanded methylation canyon encompasses a genomic region encoding transcription factors. In some embodiments, an expanded methylation canyon encompasses a genomic region encoding genes frequently dysregulated in human leukemia. In some embodiments, an expanded methylation canyon comprises HOX gene clusters (e.g., HOXA and/or HOXB gene clusters).

In some embodiments, the expanded methylation canyons associated with DNMT3A mutation are coated with H3K79me2. In some embodiments, a subject having AML associated with one or more mutations in DNMT3A can be identified by assaying a subject having AML (e.g., a biological sample obtained from a subject having AML) to determine whether increased levels of H3K79me2 are present in the sample. In some embodiments, a general increase in H3K79me2 can be detected (e.g., in a sample from the subject). In some embodiments, an increase in H3K79me2 associated with an expanded methylation canyon can be detected (e.g., in a sample from the subject). In some embodiments, an increased level of H3K79me2 is an amount that is higher than expected in a healthy sample. For example, in some embodiments an increased level is an amount that is higher than in a DNMT3A wild-type control (for example an amount in a sample from a subject that does not have a DNMT3A mutation, or an amount in a cell line that does not have a DNMT3A mutation). In some embodiments, a level of H3K79me2 in a sample can be compared to a reference amount (e.g., an amount previously measured for a DNMT3A wild-type control).

In some embodiments, the present disclosure provides a method for treating, preventing, and/or alleviating one or more symptoms of AML in a subject comprising: obtaining a sample from the subject and detecting one or more mutations in DNMT3A in the sample, wherein the presence of one or more mutations in DNMT3A indicates the subject is responsive to a DOT1L inhibitor. In some embodiments, one or more DOT1L inhibitor compounds are administered to the subject in a therapeutically effective amount.

In some embodiments, the present disclosure provides a method for treating, preventing, and/or alleviating one or more symptoms of AML in a subject comprising: obtaining a sample from the subject; detecting the presence of one or more expanded methylation canyons associated with DNMT3A in the sample; and administering to the subject a therapeutically effective amount of one or more DOT1L inhibitors when said canyons are present in the sample.

In any of the methods described herein, the sample can be selected from bone marrow, peripheral blood cells, blood, cerebrospinal fluid, skin lesions, chloroma biopsy, plasma, serum, urine, saliva, a cell, or other suitable source.

In another aspect, the disclosure provides methods of selecting a therapy for a subject having leukemia (e.g., AML). In some embodiments, a method includes detecting the presence of (a) one or more mutations in DNMT3A, and/or (b) one or more expanded methylation canyons associated with DNMT3A in a sample from the subject; and selecting, based on the presence of (a) and/or (b) in the sample, a DOT1L inhibitor for treating leukemia. Accordingly, in some embodiments, a method includes detecting the presence of one or more mutations in DNMT3A in a sample from the subject, or detecting one or more expanded methylation canyons associated with DNMT3A in a sample from the subject, or detecting the presence of one or more mutations in DNMT3A and one or more expanded methylation canyons associated with DNMT3A in a sample from the subject; and selecting, based on the presence of any of the foregoing in the sample, a DOT1L inhibitor for treating leukemia. In some embodiments, the method further includes administering to the subject a therapeutically effective amount of the DOT1L inhibitor.

In some aspects, a therapeutically effective amount of one or more DOT1L inhibitor compounds can be formulated with a pharmaceutically acceptable carrier for administration to a mammal, for example a human subject, for use in treating or preventing leukemia (e.g., AML) associated with one or more mutations in DNMT3A, and/or one or more expanded methylation canyons associated with DNMT3A mutation.

Accordingly, in certain embodiments, the compounds of the present disclosure are useful for treating, preventing, or reducing the risk of leukemia (e.g., AML) or for the manufacture of a medicament for treating, preventing, or reducing the risk of leukemia (e.g., AML). In some embodiments, compounds or formulations described herein can be administered, for example, via oral, parenteral, otic, ophthalmic, nasal, or topical routes, to provide an effective amount of the compound to the mammal. In some embodiments, compounds or formulations described herein can be administered, for example, via intravenous (IV) infusion (e.g., continuous IV infusion for several days to several weeks).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing aspects of the present disclosure, suitable methods and materials are described herein. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the disclosure will be apparent from the following detailed description and claims.

### BRIEF DESCRIPTION OF THE DRAWINGS

FIG. 1 shows a pie chart illustrating methylation canyon size dynamics in Dnmt3a knock-out mouse Hematopoietic Stem Cells (HSC). On 44% of canyons, the edges were eroded such that they increased in size, and 31% of canyons experienced hypermethylation at the edges, such they decreased in size, and 25% experienced no significant change in size.

FIG. 2 illustrates an embodiment where H3K79me2 is specifically associated with expanding methylation canyons of the HoxA gene cluster. Canyon expansion is a result of Dnmt3a loss.

FIG. 3 illustrates an embodiment where H3K79me2 is specifically associated with expanding methylation canyons of the HoxB gene cluster. Canyon expansion is a result of Dnmt3a loss.

FIG. 4 illustrates an embodiment of reduction of cell proliferation (top) and induction of apoptosis (bottom) of patient-derived AML cell lines in response to treatment with the DOT1L inhibitor SYC-522. Note that AML cell lines respond to SYC-522 treatment in a dose-dependent fashion. OCI AML2 and OCI AML3 cells bear mutations in DNMT3a (homozygous R635W and heterozygous R882C, respectively). MV4-11 is an MLL-rearranged cell line known to be sensitive to DOT1L inhibition.

FIG. 5 illustrates an embodiment where treatment of DNMT3A mutant AML cell lines OCI AML2 and OCI AML3 with DOT1L inhibitor SYC-522 induces apoptosis (top) and terminal differentiation (bottom) in a dose-dependent manner. Induction of terminal differentiation was measured by staining with CD14, a marker of cell differentiation.

FIG. 6 illustrates an embodiment where treatment of DNMT3A mutant AML cell lines OCI AML2 and OCI AML3 with the DOT1L inhibitor SYC-522 causes a reduction in H3K79 methylation in a time-dependent manner.

FIG. 7 shows a reduction in cell proliferation resulting from treatment with the DOT1L inhibitor EPZ004777 in the DNMT3A mutant AML cell lines (OCIAML2, OCIAML3(line 1) and OCIAML3 (line 2)). A significant decrease in cell proliferation was observed in all cell lines treated with 3 μM of the compound.

FIG. 8 illustrates an embodiment where apoptosis of DNMT3A mutant human AML cells is caused by treatment with 3 μM of EPZ00477. The top panel shows % of apoptotic cells marked by Annexin V binding above baseline in OCIAML2, OCIAML3 (line 1) and OCIAML3 (line 2) cells. The bottom panel shows representative flow cytometry data indicating the increase in Annexin V binding after 14 days of treatment (right peak) above untreated baseline (left peak).

FIG. 9 shows flow cytometry data of OCIAML2 and OCIAML3 cells treated with 3 μM EPZ004777 and stained for CD14, a marker of cell differentiation. A significant increase in CD14-positive cells was observed in treated groups by Day 7 and Day 9 for both cell lines.

FIG. 10 illustrates an embodiment where DOT1L inhibition reduces HOX gene expression in DNMT3A mutant cells. Bars represent from left to right: untreated, day 6, and day 10 of treatment with 3 μM EPZ004777.

FIGS. 11A-11E show an embodiment where Dnmt3a^{-/-} HSCs are characterized by increased DOT1L expression and increased H3K79 methylation that is associated with altered DNA methylation. FIG. 11A shows RNAseq data showing mRNA expression of DOT1L in murine Dnmt3aKO HSCs (bottom panel) compared to HSC from wild-type HSCs from 4-, 12- and 24-month old mice. FIG. 11B shows quantified DOT1L RNAseq data. FIG. 11C shows relative density of H3K79me2 at transcription start sites (TSS), protein coding start sites, undermethylated regions (UMRs) and DNA methylation canyons in Dnmt3aKO HSCs relative to wild-type HSCs. FIG. 11D shows relative H3K79me2 density at DNA methylation canyons that expand with Dnmt3a deletion (Dnmt3aKO HSCs; wild-type HSCs), canyons that align (Dnmt3aKO HSCs; wild-type HSCs), and canyons that shrink with Dnmt3a deletion (Dnmt3aKO HSCs; wild-type HSCs). FIG. 11E shows a representative DNA methylation canyon that expands with Dnmt3a deletion (DNA methylation; Canyon extended bar) and associated H3K79me2 in wild-type HSCs and Dnmt3aKO HSCs.

FIGS. 12A-12C illustrate an embodiment where DOT1L-induced histone 3 lysine 79 methylation is increased in DNMT3A-mutant human AML cell lines. FIG. 12A shows relative level of unmethylated H3 lysine 79. FIG. 12B shows relative level of mono-methylated H3K79. FIG. 12C shows relative level of di-methylated H3K79. Relative levels were measured by mass spectrometry in four AML cell lines KG1, THP1, OCI AML2 and OCI AML3.

FIGS. 13A-13C show an embodiment where pharmacologic DOT1L inhibition reduces cellular H3K79me and oncogenic HOX gene expression in DNMT3A mutant AML cells. FIG. 13A shows immunoblot analysis of cellular H3K79me2 in OCI AML2 and OCI AML3 cells after treatment with 3 μM EPZ004777. FIG. 13B shows relative expression determined by quantitative RT PCR of leukemogenic HOXA9, MEIS1, HOXB3, HOXB8 genes and the housekeeping gene GAPDH in OCI AML 2 cells treated with 3 μM EPZ004777 or vehicle control. FIG. 13C shows relative expression determined by quantitative RT PCR of leukemogenic HOXA9, MEIS1, HOXB3, HOXB8 genes and the housekeeping gene GAPDH in OCI AML3 cells treated with 3 μM EPZ004777 or vehicle control.

FIGS. 14A-14B show an embodiment where EPZ004777 treatment inhibits the proliferation of DNMT3A-mutant human AML cell lines in a dose- and time-dependent fashion. FIG. 14A shows growth of OCI AML2 (left panel) and OCI AML3(right panel) cells treated with increasing concentrations of EPZ004777 for 14 days. Numbers are plotted on logarithmic scale. FIG. 14B shows HL60, MV411, OCI AML2 and OCI AML3 cells treated with 3 μM EPZ004777 or vehicle control. Cells were re-plated at a constant concentration in fresh drug-containing media every 2-3 days.

FIGS. 15A-15E show one embodiment where EPZ004777 treatment induces apoptosis, cell cycle arrest and terminal differentiation in DNMT3A-mutant human AML cells in a dose-dependent manner. OCI AML2 and OCI AML3 cells were treated with increasing concentrations of EPZ004777 or vehicle control. Cells were re-plated at a constant concentration in fresh drug-containing media every 2-3 days. HL60, MV411, OCI AML2 and OCI AML3 cells were treated with 3 μM EPZ004777 or vehicle control. Cells were re-plated at a constant concentration in fresh drug-containing media every 2-3 days. FIG. 15A shows induction of apoptosis as measured every 2-3 days by Annexin V binding (AVB) flow cytometry assay of cells treated with increasing concentrations of EPZ004777. Flow histograms on days 5, 11 and 14 of treatment are shown in the left panels and quantification of % of cells AVB+ is shown in the right panels. FIG. 15B shows time dependent induction of apoptosis as measured by AVB flow cytometry assay shown as percentage of cells AVB+ minus percentage of AVB+ cells treated with vehicle control. FIG. 15C shows quantification of cell cycle analyses of OCI AML2 and OCI AML3 cells treated with increasing concentrations of EPZ004777 or vehicle control performed by flow cytometry for DNA content at specified time points. FIG. 15D shows representative flow cytometry plots of PI DNA content cell cycle analysis for OCI AML2 cells and OCI AML3 cells (left panels) with quantification in the graph on the right. FIG. 15E shows representative flow plots of CD14 cell surface expression OCI AML 2 and OCI AML 3 cells treated with vehicle control (untreated) or 3 μM EPZ004777 for 14 days (left panels) and quantification of percentage of CD14⁺ cells treated with 3 μM EPZ004777 or vehicle control at specified time points. Error bars represent standard deviation.

FIGS. 16A-16C illustrate in vivo efficacy of pharmacologic DOT1L inhibition in DNMT3A-mutant AML. H3K79me2 levels as measured by ELISA measured in OCI AML3 subcutaneous tumors (left) and bone marrow from vehicle control-treated animals or animals treated with 35 or 70 mg/kg/day EPZ5676 administered via continuous IV infusion (right). FIG. 16B shows volume of OCI AML3 subcutaneous tumors over time in vehicle control treated animals and animals treated with 35 or 70 mg/kg/day EPZ5676 administered via continuous IV infusion for 21 days. FIG. 16C shows relative expression of MEIS1 and HOXB3 in OCI AML3 subcutaneous tumors in vehicle control treated mice and mice treated with 35 or 70 mg/kg/day EPZ5676 for 21 days.

FIGS. 17A-17D show an embodiment where DOT1L inhibitor treatment selectively inhibits the in vitro growth and induces terminal differentiation of primary patient samples with DNMT3A mutations. FIG. 17A shows relative colony forming units (CFU) of normal cord blood CD34⁺ cells and primary AML samples wild-type for both DNMT3A and MLL (left panel), primary AML samples with MLL anomalies (middle panel), and primary AML samples with DNMT3A mutations (right panel) treated with vehicle control or 3 μM EPZ004777. Assays performed in triplicate, error bars represent standard deviation. Patient numbers on horizontal axis correspond to patient numbers in Table 1. FIG. 17B shows average change in CFC of primary patient samples treated with EPZ004777 compared to vehicle treated control. FIG. 17C shows flow cytometry analysis of CD14 expression of primary AML cells with DNMT3A mutation isolated from plates after treatment with vehicle control or 3 μM EPZ004777. FIG. 17D shows H&E staining of primary AML cells with DNMT3A mutation isolated from plates after treatment with vehicle control or 3 μM EPZ004777.

FIGS. 18A-18B illustrate an embodiment where SYC-522 inhibits growth and induces apoptosis of DNMT3A-mutant AML cell lines in a dose-dependent manner. OCI AML2 and OCI AML3 cells were treated with increasing concentrations of SYC-522 or vehicle control. Cells were re-plated at a constant concentration in fresh drug-containing media every 3-4 days. FIG. 18A shows growth curves of cells treated with SYC-522 or vehicle control for 10 days. Numbers are plotted on logarithmic scale. FIG. 18B shows induction of apoptosis as measured on days 7 and 10 of treatment with SYC-522 or vehicle control by Annexin V binding (AVB) flow cytometry assay.

FIG. 19 shows an embodiment where EPZ004777 treatment of primary AML patient samples (patient numbers corresponding to patient numbers shown in Table 1) with DNMT3A mutation induces terminal differentiation. Flow cytometry plots of primary AML primary patient samples isolated from culture plates after treatment in CFC assay with vehicle control (untreated) or 3 μM EPZ004777 are shown. Plots show live (PI negative) CD45+ cells.

### DETAILED DESCRIPTION

Aspects of the present disclosure are based in part upon the surprising discovery that DOT1L inhibitors can effectively treat acute myeloid leukemia (AML) associated with one or more DNMT3A mutations.

In some embodiments, leukemia cells having one or more mutations in DNMT3A are sensitive to the DOT1L inhibitors as described herein. Accordingly, the present disclosure provides methods of treating, preventing, or alleviating one or more symptoms of leukemia associated with one or more mutations in DNMT3A in a subject by administering a therapeutically effective amount of a DOT1L inhibitor to the subject. In some embodiments, the present disclosure provides methods of treating, preventing, or alleviating one or more symptoms of leukemia in a subject having one or more expanded methylation canyons associated with DNMT3A mutation by administering a therapeutically effective amount of a DOT1L inhibitor to the subject. In some embodiments, the present disclosure provides methods of treating, preventing, or alleviating one or more symptoms of AML associated with one or more expanded methylation canyons in a subject as a result of DNMT3A mutation(s), wherein the expanded methylation canyons comprise HOX gene clusters, by administering a therapeutically effective amount of a DOT1L inhibitor to the subject. In some embodiments, the present disclosure provides methods of treating, preventing or alleviating one or more symptoms of AML associated with increased levels of H3K79me2. In some embodiments, increased levels of H3K79me2 result in H3K79me2 coating expanded methylation canyons associated with DNMT3A mutation(s).

As used herein, “methylation canyon” refers to a location in the genome of a subject (e.g., in cells of the subject, for example in hematopoietic stem cells of the subject) comprising expansive regions lacking DNA methylation (e.g., one or more hypomethylated DNA regions). In some embodiments, the methylation canyons described herein are greater than 3.5kb in length and have a methylation ratio of less than 0.1. In some embodiments, the methylation canyons are enriched in genes that encode proteins involved in transcription regulation and/or in genes encoding a homeobox domain. In some embodiments, mutation(s) of DNMT3A cause(s) alterations in the size of methylation canyons. In some embodiments, mutation(s) of DNMT3A cause(s) the expansion of methylation canyons. In some embodiments, the expanded methylation canyons are coated with a higher amount of H3K79me2 than a corresponding cell that is positive for DNMT3A activity. In some embodiments, the expanding canyons coated with H3K79me2 comprise one or more HOX gene clusters.

Aspects of the disclosure are particularly useful for treating certain forms of AML that have a poor prognosis. Mutations of DNMT3A in AML typically predict a poor prognosis. According to aspects of the disclosure, mutations in DNMT3A induce an aggressive myeloid leukemia that can be treated with one or more DOT1L inhibitors.

In some embodiments, DOT1L inhibitor compounds described herein inhibit the histone methyltransferase activity of DOT1L or a mutant thereof and are useful to treat certain forms of AML. Based upon the surprising discovery that methylation regulation (e.g., histone methylation regulation) by DOT1L is involved in progression of AML bearing mutations in DNMT3A, the compounds described herein are useful for treating certain forms of acute myeloid leukemia.

In some embodiments, the present disclosure features a method for treating or alleviating one or more symptoms of DNMT3A-deficient AML (e.g., AML bearing one or more mutations in DNMT3A). The method includes administering to a subject in need thereof, a therapeutically effective amount of a DOT1L inhibitor or a pharmaceutically acceptable salt, prodrug, metabolite, polymorph, solvate, or stereoisomer thereof.

In some aspects, the disclosure relates to DNMT3A-deficient AML (e.g., AML bearing one or more mutations in DNMT3A). In some embodiments, DNMT3A-deficient AML is responsive to treatment with DOT1L inhibitors. In some embodiments, the present disclosure provides methods for the treatment of DNMT3A-deficient AML (e.g., to prevent or slow disease progression and/or to kill diseased cells) in a subject in need thereof by administering to a subject in need of such treatment, a therapeutically effective amount of a compound of the present disclosure (e.g., a DOT1L inhibitor), or a pharmaceutically acceptable salt, prodrug, metabolite, polymorph or solvate thereof. The present disclosure further provides the use of one or more DOT1L inhibitors, or a pharmaceutically acceptable salt, prodrug, metabolite, polymorph or solvate thereof, for the preparation of a medicament useful for the treatment of DNMT3A-deficient AML (e.g., in a human subject).

In some embodiments, the present disclosure provides methods for the treatment of a DNMT3A-deficient AML, the course of which is influenced by modulating the methylation status of histones or other proteins, wherein said methylation status is mediated at least in part by the activity of DOT1L.

Modulation of the methylation status of histones can in turn influence the level of expression of target genes activated by methylation, and/or target genes suppressed by methylation. The method includes administering to a subject in need of such treatment, a therapeutically effective amount of a DOT1L inhibitor as described herein, or a pharmaceutically acceptable salt, prodrug, metabolite, polymorph, solvate, or stereoisomer thereof.

In one aspect, methods described herein are useful to treat leukemia. In some embodiments, the leukemia is acute myeloid leukemia (AML). AML is a cancer of the myeloid line of blood cells characterized by the abnormal growth of white blood cells that accumulate in the bone marrow and interfere with the production of normal blood cells. AML has several subtypes. In some aspects, the instant disclosure relates to the subtype of AML associated with mutation(s) in DNMT3A (e.g., DNMT3A-deficient AML). Mutation of DNMT3A is known to occur at several locations of the gene. In some embodiments, mutations of the gene encoding DNMT3A cause a change in the amino acid sequence of the DNMT3A protein. Non-limiting examples of locations where DNMT3A mutations are known to occur are M315fs (frameshift), K468R, E477(stop), E505(stop), Q515(stop), G590(frameshift), Q615(stop), E616(frameshift), P718L, L723(frameshift), R729Q, R729W, R736H, A741V, L773(deletion), R792H, R805S, K829R, K841Q, R882P, R882H, R882C, and F909C. In some embodiments, other mutations are present in addition to mutations of DNMT3A. In some embodiments, other mutations co-occur with mutation of DNMT3A. In some embodiments, the co-occurring mutations are NPMc+, FLT3/ITD, IDH1/2, MLL_PTD, and/or mutations of the cohesion complex.

In some aspects, mutations of DNMT3A are associated with types of cancer other than AML. Non-limiting examples of other cancers (e.g., other haem malignancies) associated with mutations of DNMT3A are Primary Myelo-Fibrosis, CMML, MDS, T-cell lymphoma, T-ALL, ETP-ALL, juvenile MML, mastocytosis, and MPAL.

The present disclosure further provides the use of a compound described herein, or a pharmaceutically acceptable salt, ester, prodrug, metabolite, polymorph or solvate thereof in the treatment of DNMT3A-deficient AML, or, for the preparation of a medicament useful for the treatment of such DNMT3A-deficient AML.

Compounds of the present disclosure can selectively inhibit proliferation of leukemia cells characterized by mutation(s) of DNMT3A.

Accordingly, the present disclosure provides methods for treating or alleviating a symptom of DNMT3A-deficient AML characterized by the presence of expanded methylation canyons associated with H3K79me2 by a compound of the present disclosure, or a pharmaceutically acceptable salt, ester, prodrug, metabolite, polymorph or solvate thereof.

The present disclosure also provides methods for treating or alleviating a symptom of DNMT3A-deficient AML characterized by the presence of expanded methylation canyons associated with H3K79me2. For example, in some embodiments a method comprises obtaining sample from a subject, detecting the presence of one or more mutations in DNMT3A in the sample, and when mutation is present in the sample, administering to the subject a therapeutically effective amount of a DOT1L inhibitor

In other aspects, the present disclosure provides personalized medicine, treatment and/or AML management for a subject by genetic screening for mutations in DNMT3A in the subject. For example, the present disclosure provides methods for treating, preventing or alleviating a symptom of leukemia or a precancerous condition by determining responsiveness of the subject to a DOT1L inhibitor and when the subject is responsive to the DOT1L inhibitor, administering to the subject a therapeutically effective amount of the DOT1L inhibitor, or a pharmaceutically acceptable salt, prodrug, metabolite, polymorph, solvate, or stereoisomer thereof. In some embodiments, the responsiveness can be determined by obtaining a sample from the subject and detecting one or more mutations in DNMT3A, and the presence of such mutations indicates that the subject is responsive to the DOT1L inhibitor. Once the responsiveness of a subject is determined, a therapeutically effective amount of a DOT1L inhibitor can be administered. The therapeutically effective amount of a DOT1L inhibitor can be determined by one of ordinary skill in the art.

In other aspects, the present disclosure provides personalized medicine, treatment and/or cancer management for a subject by genetic screening of methylation canyons. In some aspects, AML subtypes are associated with particular expanded methylation canyons, including expanded methylation canyons coated with a higher amount of H3K79me2 than a corresponding cell that is positive for DNMT3A activity. In some aspects, the expanded methylation canyons coated with H3K79me2 comprise HOX gene clusters.

As used herein, the term “responsiveness” is interchangeable with terms “responsive”, “sensitive”, and “sensitivity”, and it is meant that a subject shows one or more therapeutic responses when administered an DOT1L inhibitor, e.g., leukemia cells or leukemia progenitor cells of the subject undergo apoptosis and/or necrosis, differentiation and/or display reduced growth, division, or proliferation. This term can also mean that a subject will or has a higher probability, relative to the population at large, of having a therapeutic response when administered an DOT1L inhibitor, e.g., leukemia cells or leukemia progenitor cells of the subject undergo apoptosis and/or necrosis, differentiation and/or display reduced growth, division, or proliferation.

As used herein, a “subject” is interchangeable with a “subject in need thereof”, both of which refers to a subject having DNMT3A-deficient AML that involves DOT1L-mediated protein methylation, or a subject having an increased risk of developing such a disorder relative to the population at large. A subject in need thereof may be a subject having a DNMT3A-deficient AML. A subject in need thereof can have a precancerous condition. In some embodiments, a subject in need thereof has leukemia. A subject in need thereof can have leukemia associated with DOT1L, for example AML. A subject in need thereof can have AML associated with one or more mutations in DNMT3A. A subject in need thereof can have DNMT3A-deficient AML associated with expanded methylation canyons associated with H3K79me2.

As used herein, a “subject” includes a mammal. The mammal can be, e.g., a human or appropriate non-human mammal, such as a primate, mouse, rat, dog, cat, cow, horse, goat, camel, sheep or a pig. The subject can also be a bird or fowl. In one embodiment, the mammal is a human. A subject can be male or female.

A subject in need thereof can be one who has been previously diagnosed or identified as having leukemia or a precancerous condition. A subject in need thereof can also be one who is having (suffering from) leukemia or a precancerous condition. Alternatively, a subject in need thereof can be one who has an increased risk of developing such disorder relative to the population at large (e.g., a subject who is predisposed to developing such disorder relative to the population at large).

Optionally a subject in need thereof has already undergone, is undergoing or will undergo, at least one therapeutic intervention for the leukemia or precancerous condition prior to the treatment with a DOT1L inhibitor.

A subject in need thereof may have refractory leukemia after their most recent therapy.

“Refractory leukemia” means leukemia that does not respond to treatment. The leukemia may be resistant at the beginning of treatment or it may become resistant during treatment.

Refractory leukemia is also called resistant leukemia. In some embodiments, the subject in need thereof has leukemia recurrence following remission on most recent therapy. In some embodiments, the subject in need thereof received and failed all known effective therapies for cancer treatment. In some embodiments, the subject in need thereof received at least one prior therapy.

In some embodiments, a subject in need thereof may have a secondary leukemia as a result of a previous therapy. “Secondary leukemia” means leukemia that arises after, due to, or as a result from previous carcinogenic therapies, such as chemotherapy. In some embodiments, the secondary leukemia is AML. In some embodiments, the secondary leukemia is DNMT3A-deficient AML.

In some embodiments, the mutations and/or chromosomal alterations referred to herein are somatic mutations or alterations. The term “somatic” mutation or alteration refers to a mutation or alteration (e.g., deleterious) in at least one gene allele (e.g., one or both alleles or copies of a chromosomal region) that is not found in every cell of the body, but is found only in isolated cells. A characteristic of the somatic changes as used herein is, that they are restricted to particular tissues or even parts of tissues or cells within a tissue and are not present in the whole organism harboring the tissues or cells. The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene.

Accordingly, an increase in mRNA or protein expression and/or activity levels can be detected using any suitable method available in the art. For example, an increase in activity level can be detected by measuring the biological function of a gene product (e.g., activity of DOT1L) or transcriptional activity (e.g., expression levels of target genes can be assayed using RT-PCR or other suitable technique). In some embodiments, genetic modifications (e.g., one or more mutations of DNMT3A) are associated with expanded methylation canyons and may be detected by a sequencing analysis. In some embodiments, expanded methylation canyons may be detected by methods including, but not limited to, whole genome bisulfite sequencing, methylation-specific-PCR, or Methylated DNA immunoprecipitation (MeDIP). Methods can entail the steps of genomic DNA purification, PCR amplification to amplify the region of interest, cycle sequencing, sequencing reaction cleanup, capillary electrophoresis, and/or data analysis. Alternatively or in addition, a method may include the use of microarray-based targeted region genomic DNA capture and/or sequencing.

In some embodiments, levels of H3K79me2 can be determined by histone extraction followed by Western blotting or bisulfite sequencing. However, it should be appreciated that other techniques can be used.

Kits, reagents, and methods for selecting appropriate PCR primers and performing resequencing are commercially available, for example, from Applied Biosystems, Agilent, and NimbleGen (Roche Diagnostics GmbH). Detection of mRNA expression can be detected by methods known in the art, such as Northern blot, nucleic acid PCR, quantitative RT-PCR, expression array or RNA-sequencing. Detection of polypeptide expression (e.g., wild-type or mutant) can be carried out with any suitable immunoassay in the art, such as Western blot analysis.

By “sample” is meant any biological sample derived from the subject, includes but is not limited to, cells, tissues samples, body fluids (including, but not limited to, mucus, blood, plasma, serum, urine, saliva, and semen), cancer cells, and cancer tissues. In some embodiments, the sample is selected from bone marrow, peripheral blood cells, blood, cerebrospinal fluid, skin lesions, chloroma biopsies, plasma and serum. In some embodiments, a sample consists of or contains leukemic blasts from a patient that has a hematologic malignancy.

Samples can be provided by the subject under treatment or testing. Alternatively samples can be obtained by the physician according to routine practice in the art.

The present disclosure also provides methods for determining predisposition of a subject to DNMT3A-deficient AML by obtaining a sample from the subject and detecting one or more mutations in DNMT3A. In some embodiments, the presence of such mutations can be used to indicate that the subject is predisposed to (e.g., has a higher risk of) developing leukemia compared to a subject without such mutations.

The term “predisposed” as used herein in relation to leukemia or a precancerous condition is to be understood to mean the increased probability (e.g., at least 1%, 5%, 10%, 20%, 30%, 40%, 50%, 60%, 70%, 80%, 90%, 100%, 150%, 200%, or more increase in probability) that a subject with one or more mutations in DNMT3A, will suffer leukemia, as compared to the probability that another subject not having one or more mutations in DNMT3A, will suffer leukemia, under circumstances where other risk factors (e.g., chemical/environment, food, and smoking history, etc.) for having leukemia between the subjects are the same.

“Risk” in the context of the present disclosure, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

In other example, the present disclosure provides methods of AML management in a subject by determining predisposition of the subject to DNMT3A-deficient AML periodically. The methods comprise steps of obtaining a sample from the subject and detecting one or more mutations in DNMT3A, and the presence of such mutation(s) indicates that the subject is predisposed to developing DNMT3A-deficient AML compared to a subject without such mutations in DNMT3A.

As used herein, the term “acute myeloid leukemia (AML)” refers to a cancer of the myeloid line of blood cells characterized by the abnormal growth of white blood cells that accumulate in the bone marrow and interfere with the production of normal blood cells. AML has several subtypes. In some aspects, the instant disclosure relates to the subtype of AML associated with mutations in DNMT3A (DNMT3A-deficient AML). In some aspects, AML subtypes are associated with the presence of particular methylation canyons. In some aspects, the expanded methylation canyons are coated with a higher amount of H3K79me2 than a corresponding cell that is positive for DNMT3A activity. In some embodiments, the expanded methylation canyons associated with H3K79me2 comprise HOX gene clusters.

As used herein, “treating” or “treat” describes the management and care of a patient for the purpose of combating a disease, condition, or disorder and includes the administration of a compound of the present disclosure, or a pharmaceutically acceptable salt, prodrug, metabolite, polymorph or solvate thereof, to alleviate the symptoms or complications of a disease, condition or disorder, or to eliminate the disease, condition or disorder.

A compound of the present disclosure, or a pharmaceutically acceptable salt, prodrug, metabolite, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as leukemia, which can occur in multiple locations, is alleviated if the severity of the leukemia is decreased within at least one of multiple locations.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals. As used herein the term “sign” is also defined as an indication that something is not right in the body. But signs are defined as things that can be seen by a doctor, nurse, or other health care professional.

Treating or preventing a leukemia can result in a reduction in the rate of leukemia cell or leukemia progenitor cell proliferation. Preferably, after treatment, the rate of leukemia-associated cell proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time. The rate of cellular proliferation may also be measured by any method commonly known in the art, for example flow cytometry.

Treating or preventing a leukemia can result in an increase in the rate of normal blood cell proliferation. Preferably, after treatment, the rate of normal blood cell proliferation is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time. The rate of cellular proliferation may also be measured by any method commonly known in the art, for example flow cytometry.

Treating or preventing a leukemia can result in a reduction in the proportion of proliferating leukemia cells or leukemia progenitor cells. Preferably, after treatment, the proportion of proliferating leukemia cells or leukemia progenitor cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a leukemia can result in an increase in the proportion of normal blood cells. Preferably, after treatment, the proportion of proliferating normal cells is increased by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating normal cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing leukemia can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of excessive accumulation of immature cells (blasts) and differentiation arrest, or disordered (dysplastic) differentiation.

Treating leukemia, for example AML, can result in leukemia cell death, and preferably, leukemia cell death results in a decrease of at least 10% in number of leukemia cells in a population. More preferably, leukemia cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci U S A. 100(5): 2674-8, 2003. In an aspect, leukemia cell death occurs by apoptosis.

Treating leukemia, for example AML, can result in leukemia cell differentiation, and preferably, leukemia cell differentiation results in a decrease of at least 10% in number of undifferentiated leukemia cells (leukemic blasts) in a population. More preferably, leukemia cell differentiation means a decrease of at least 20%; more preferably, a decrease of at least 30%; more preferably, a decrease of at least 40%; more preferably, a decrease of at least 50%; most preferably, a decrease of at least 75%. The number of cells in a population may be measured by any reproducible means. The number of blasts and differentiated cells in a population can be measured by fluorescence activated cell sorting (FACS), immunofluorescence microscopy and light microscopy.

In some embodiments, an effective amount of a compound of the present disclosure, or a pharmaceutically acceptable salt, prodrug, metabolite, polymorph or solvate thereof, is not significantly cytotoxic to normal cells. A therapeutically effective amount of a compound is not significantly cytotoxic to normal cells if administration of the compound in a therapeutically effective amount does not induce normal cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present disclosure, or a pharmaceutically acceptable salt, prodrug, metabolite, polymorph or solvate thereof, can induce or activate cell death selectively in AML cells. Administering to a subject in need thereof a compound of the present disclosure, or a pharmaceutically acceptable salt, prodrug, metabolite, polymorph or solvate thereof, can induce or activate cell death selectively in AML cells. Contacting a cell with a compound of the present disclosure, or a pharmaceutically acceptable salt, prodrug, metabolite, polymorph or solvate thereof, can induce cell death selectively in one or more cells affected by AML. Preferably, administering to a subject in need thereof a compound of the present disclosure, or a pharmaceutically acceptable salt, prodrug, metabolite, polymorph or solvate thereof, induces cell death selectively in one or more cells affected by AML.

In some embodiments, the present disclosure relates to a method of treating or preventing AML by administering a compound of the present disclosure, or a pharmaceutically acceptable salt, prodrug, metabolite, polymorph or solvate thereof, to a subject in need thereof, where administration of the compound of the present disclosure, or a pharmaceutically acceptable salt, prodrug, metabolite, polymorph or solvate thereof, results in one or more of the following: accumulation of cells in G1 and/or S phase of the cell cycle, cytotoxicity via cell death in AML cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N. Y.; Enna et al., Current Protocols in Pharmacology, John Wiley & Sons, N. Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

As used herein, a DOT1L inhibitor is an inhibitor of DOT1L-mediated protein methylation (e.g., an inhibitor of histone methylation). In some embodiments, a DOT1L inhibitor is a small molecule inhibitor of DOT1L. In some embodiments, a DOT1L inhibitor is a compound of formula:

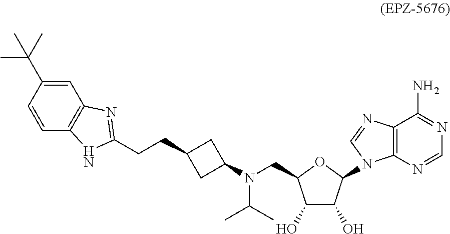 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, prodrug, metabolite, polymorph, solvate, or stereoisomer thereof.

In some embodiments, a DOT1L inhibitor is a compound of formula:

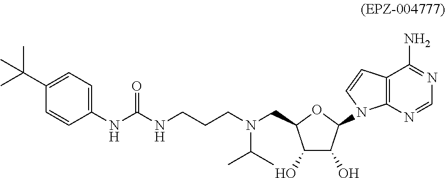 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or a pharmaceutically acceptable salt, prodrug, metabolite, polymorph, solvate, or stereoisomer thereof.

In some embodiments, a DOT1L inhibitor is a compound of formula:

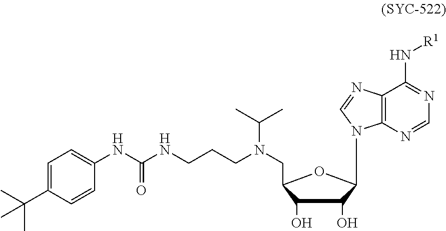

wherein R1 is a H, or a pharmaceutically acceptable salt, prodrug, metabolite, polymorph, solvate or stereoisomer thereof.

Other DOT1L inhibitors suitable for use according to methods described herein are provided in WO2012/075381, WO2012/075492, WO2012/082436, WO2012/75500, WO2014/026198, WO2014/035140, US2014/0100184, and in J. Med Chem. (2013), 56: p. 8972-8983, the contents of each of which are hereby incorporated by reference in their entirety. The activity of a DOT1L inhibitor can be evaluated in an assay, for example by comparing the histone methyltransferase activity of DOT1L (e.g., methylation of histone substrates such as H3K79 by immunoblot) in the presence or absence of different amounts of the inhibitor.

The disclosure also relates to a pharmaceutical composition of a therapeutically effective amount of a DOT1L inhibitor disclosed herein and a pharmaceutically acceptable carrier.

The disclosure also relates to a pharmaceutical composition of a therapeutically effective amount of a salt of a DOT1L inhibitor disclosed herein and a pharmaceutically acceptable carrier.

The disclosure also relates to a pharmaceutical composition of a therapeutically effective amount of a hydrate of a DOT1L inhibitor disclosed herein and a pharmaceutically acceptable carrier.

The present disclosure also relates to use of the compounds disclosed herein in preparation of a medicament for treating or preventing leukemia. The use includes a DOT1L inhibitor disclosed herein for administration to a subject in need thereof in a therapeutically effective amount. The leukemia can be AML. In some embodiments, the AML is DNMT3A-deficient AML. In some embodiments, the DNMT3A-deficient AML is associated with one or more expanded methylation canyons. In some embodiments, the expanded methylation canyons are coated with a higher amount of H3K79me2 than a corresponding cell that is positive for DNMT3A activity. In some embodiments, the expanded methylation canyons coated with H3K79me2 comprise HOX gene clusters.

In some embodiments, compounds provided herein can be formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered continuously (e.g., intravenously) three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations). In some embodiments the administration regimen is a continuous IV infusion (e.g., 24 hours per day) for one or more weeks (e.g., 1-4, 4-8, or longer), for example a 28-day continuous IV infusion of each 28-day cycle.

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It should be appreciated that in some embodiments, a DOT1L inhibitor compound or composition can be administered as a monotherapy. As used herein, “monotherapy” refers to the administration of a single active or therapeutic compound to a subject in need thereof. In some embodiments, monotherapy will involve administration of a therapeutically effective amount of a single active compound, for example, AML monotherapy with one of the DOT1L inhibitor compounds described herein, or a pharmaceutically acceptable salt, prodrug, metabolite, analog or derivative thereof, to a subject in need of treatment of AML. In one aspect, the single active DOT1L inhibitor compound is a compound described herein, or a pharmaceutically acceptable salt, prodrug, metabolite, polymorph or solvate thereof.

It will be appreciated that in some embodiments, two or more DOT1L inhibitor compounds can be administered to a subject (e.g., to treat AML).

It also will be appreciated that in some embodiments one or more DOT1L inhibitor compounds or compositions, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

In some embodiments, a DOT1L inhibitor compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U. S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, an additional therapeutically active agent is an AML standard of care agent. In certain embodiments, an additional therapeutically active agent is Ara-C, or daunorubicin. In certain embodiments, an additional therapeutically active agent is a DNA methyltransferase inhibitor. In certain embodiments, an additional therapeutically active agent is azacitidine or decitabine. In certain embodiments, an additional therapeutically active agent is a histone deacetylase inhibitor. In certain embodiments, an additional therapeutically active agent is vorinostat or panobinostat. In certain embodiments, an additional therapeutically active agent is a demethylase inhibitor. In certain embodiments, an additional therapeutically active agent is tranylcypromine or LSD1 inhibitor II. In certain embodiments, an additional therapeutically active agent is a bromodomain inhibitor. In certain embodiments, an additional therapeutically active agent is IBET-151 or JQ1. In certain embodiments, an additional therapeutically active agent is an ALL standard of care agent. In certain embodiments, an additional therapeutically active agent is mitoxantrone, methotrexate, mafosfamide, prednisolone, or vincristine.

In certain embodiments, an additional therapeutically active agent is prednisolone, dexamethasone, doxorubicin, vincristine, mafosfamide, cisplatin, carboplatin, Ara-C, rituximab, azacitadine, panobinostat, vorinostat, everolimus, rapamycin, ATRA (all-trans retinoic acid), daunorubicin, decitabine, Vidaza, mitoxantrone, or IBET-151.

It also should be appreciated that in some embodiments, a DOT1L inhibitor compound or composition can be administered in conjunction with chemotherapy, radiation therapy, and/or a cytostatic agent. In some embodiments, treatment methods described herein are administered in conjunction with anti-VEGF or anti-angiogenic factor, and/or p53 reactivation agent. Non-limiting examples of cancer chemotherapeutic agents include, but are not limited to, irinotecan (CPT-11); erlotinib; gefitinib (Iressa®); imatinib mesylate (Gleevec®); oxalipatin; anthracyclins-idarubicin and daunorubicin; doxorubicin; alkylating agents such as melphalan and chlorambucil; cis-platinum, methotrexate, and alkaloids such as vindesine and vinblastine. A cytostatic agent is any agent capable of inhibiting or suppressing cellular growth and multiplication. Non-limiting examples of cytostatic agents include paclitaxel, 5-fluorouracil, 5-fluorouridine, mitomycin-C, doxorubicin, and zotarolimus. Other cancer therapeutics that can be used in conjunction with a DOT1L inhibitor include inhibitors of matrix metalloproteinases such as marimastat, growth factor antagonists, signal transduction inhibitors and protein kinase C inhibitors. In some embodiments, methods described herein can be used in combination with treatment options such immunotherapy and/or cancer vaccines.

It should be appreciated that in some embodiments, the term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies.

The present disclosure also provides pharmaceutical compositions comprising one or more DOT1L inhibitor compounds described herein, and optionally one or more additional agents described herein, in combination with at least one pharmaceutically acceptable excipient or carrier.

A “pharmaceutical composition” is a formulation containing one or more DOT1L inhibitor compounds in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition described herein can be administered to a subject in many of the well-known methods currently used for chemotherapeutic treatment. For example, for treatment of leukemia, a DOT1L inhibitor compound or formulation may be injected directly into the blood stream or body cavities or taken orally or applied through the skin with patches. The dose chosen should be sufficient to constitute effective treatment but not as high as to cause unacceptable side effects. The state of the disease condition (e.g., leukemia, for example, AML) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician. In some embodiments, the disease or condition to be treated is leukemia (e.g., AML, for example DNMT3A-deficient AML).

For a DOT1L inhibitor compound or formulation, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms described herein are dictated by and directly dependent on the unique characteristics of the active compound and the particular therapeutic effect to be achieved.

In therapeutic applications, the dosages of the pharmaceutical compositions used as described herein vary depending on the agent or combination of agents, the age, weight, and clinical condition of the recipient patient, and the experience and judgment of the clinician or practitioner administering the therapy, among other factors affecting the selected dosage. Generally, the dose should be sufficient to result in slowing, and preferably regressing, the proliferation of leukemia cells and also preferably causing complete regression of the leukemia. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m , and age in years). An effective amount of a pharmaceutical agent is that which provides an objectively identifiable improvement as noted by the clinician or other qualified observer. For example, regression of leukemia in a patient may be measured with reference to the number of leukemia cells or leukemia precursor cells. Decrease in the number of leukemia cells indicates regression. Regression is also indicated by failure of leukemia cells to reoccur after treatment has stopped. As used herein, the term “dosage effective manner” refers to amount of an active compound to produce the desired biological effect in a subject or cell.

The compounds of the present disclosure are capable of further forming salts.

As used herein, “pharmaceutically acceptable salts” refer to derivatives of the compounds described herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds described herein can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds described herein can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p 1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds can be found in Remington: the Science and Practice of Pharmacy, 19th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing aspects of the present disclosure. The examples do not limit the claimed invention. Based on the present disclosure the skilled artisan can identify and employ other components and methodology useful for practicing aspects of the present invention.

For the compounds described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers.

The pharmaceutical compositions can be included in a container, pack, or dispenser together with instructions for administration. Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the invention remains operable.

Moreover, two or more steps or actions can be conducted simultaneously.

Compounds suitable for the methods of the disclosure, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

To further assess a compound's drug-like properties, measurements of inhibition of cytochrome P450 enzymes and phase II metabolizing enzyme activity can also be measured either using recombinant human enzyme systems or more complex systems like human liver microsomes. Further, compounds can be assessed as substrates of these metabolic enzyme activities as well. These activities are useful in determining the potential of a compound to cause drug-drug interactions or generate metabolites that retain or have no useful antimicrobial activity.

To get an estimate of the potential of the compound to be orally bioavailable, one can also perform solubility and Caco-2 assays. The latter is a cell line from human epithelium that allows measurement of drug uptake and passage through a Caco-2 cell monolayer often growing within wells of a 24-well microtiter plate equipped with a 1 micron membrane. Free drug concentrations can be measured on the basolateral side of the monolayer, assessing the amount of drug that can pass through the intestinal monolayer. Appropriate controls to ensure monolayer integrity and tightness of gap junctions are needed. Using this same system one can get an estimate of P-glycoprotein mediated efflux. P-glycoprotein is a pump that localizes to the apical membrane of cells, forming polarized monolayers. This pump can abrogate the active or passive uptake across the Caco-2 cell membrane, resulting in less drug passing through the intestinal epithelial layer. These results are often done in conjunction with solubility measurements and both of these factors are known to contribute to oral bioavailability in mammals. Measurements of oral bioavailability in animals and ultimately in man using traditional pharmacokinetic experiments will determine the absolute oral bioavailability.

Experimental results can also be used to build models that help predict physical-chemical parameters that contribute to drug-like properties. When such a model is verified, experimental methodology can be reduced, with increased reliance on the model predictability.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are meant to illustrate, but in no way to limit, the claimed invention.

### Example 1

### Materials and Methods

### Hematopoietic Stem Cell Purification and Flow Cytometry

For WT HSCs, whole bone marrow cells were isolated from femurs, tibias, pelvis and humerus of 12 month-old male C57B1/6 mice. 10 mice were used to purify HSCs; biological replicates were performed with two separate pools of HCSs from different donors. Dnmt3a-KO HSCs were purified from mice at the tertiary stage of serial transplantation, because at this point, the phenotype resulting from loss of Dnmt3a manifests most significantly. 18-weeks after the tertiary transplants, donor cell derived (CD45.2+) HSCs were purified from four to eight transplanted mice per biological replicate. This timing allowed aged-matched comparison to 12-month-old wild-type HSCs. HSCs from both WT and Dnmt3a KO mice were purified using the side population (SP) strategy of Hoechst staining in combination with surface markers. Briefly, whole bone marrow cells were resuspended in staining media at 106 cells/mL and incubated with 5 mg/mL Hoechst 33342 (Sigma) for 90 minutes at 37° C. For antibody staining, cells were suspended at a concentration of 108 cells/mL and incubated in 4° C. for 15 minutes with the desired antibodies. Magnetic enrichment was performed with c-Kit-biotin antibody(eBioscience, San Diego, Calif.) and anti-biotin microbeads (Miltenyi Biotec, Auburn, Calif.) oranti-mouse CD117 microbeads (Miltenyi Biotec, Germany) on an AutoMACS (MiltenyiBiotec, Germany). Post-enrichment, the positive cell fraction was labeled with antibodies to identify HSCs (SP+Lineage−Sca-1+c-Kit+CD150+). All antibodies were obtained from BD Biosciences (San Jose, Calif.) or eBioscience (San Diego, Calif.) and used at 1:100 dilutions. Cell sorting was performed on a MoFlo cell sorter (Dako North America, Carpinteria, Calif.) or Aria II (BD Biosciences, San Jose, Calif.) and analysis performed on a LSRII (BDBiosciences, San Jose, Calif.). All animal work was performed with approval from the Baylor College of Medicine Institutional Animal Care and Use Committee.

### Whole-Genome Bisulfite Sequencing (WGBS)

For WGBS library construction, 300 ng genomic DNA was isolated from HSCs and fragmented using a Covaris sonication system (Covaris S2). Following fragmentation, libraries were constructed using the Illumina TruSeq DNA sample preparation kit. After ligation, libraries were bisulfite-treated using the EpiTect Bisulfite Kit (Qiagen, Valencia, Calif.). Ligation efficiency tested by PCR using TrueSeq primers and Pfu TurboCx hotstar tDNA polymerase (Stratagene). After determining the optimized PCR cycle number for each sample, a large scale PCR reaction (100 ul) was performed as described previously. PCR products were sequenced with Illumina HiSeq sequencing systems.

Anti-CMS Technique for Detection of 5-hydroxymethylcytosine

For CMS precipitation, 1.5 μg of genomic DNA fragments were ligated with methylated adaptors and treated with sodium bisulfite (Qiagen). The DNA was then denatured for 10 min at 95° C. (0.4 M NaOH, 10 mM EDTA), neutralized by addition of cold 2 M ammonium acetate pH 7.0, incubated with anti-CMS antiserum in 1×immunoprecipitation buffer (10 mM sodium phosphate pH 7.0, 140 mM NaCl, 0.05% Triton X-100) for 2 h at 4° C., and then precipitated with Protein G beads. Precipitated DNA was eluted with Proteinase K, purified by phenol-chloroform extraction, and amplified by 8 cycles PCR using PfuTurboCx hotstart DNA polymerase (Stratagene). DNA sequencing was carried out using Illumina/Solexa Genome Analyzer II and HiSeq sequencing systems.

### OxBS Sequencing

Genomic DNA was further purified by ethanol precipitation and micro Bio-Spin 6 column(Bio-Rad). 250 ng purified genomic DNA was denatured in at 24 μL of 0.05 M NaOH at 37° C. for 30 min, and then snap cooled on ice for 5 min. Next, 1 μL of KRuO4 (Sigma) (15 mM in 0.05 M NaOH) was added to denatured gnomic DNA on ice for 1 hour, with occasional vortexing. The mixture was purified with micro Bio-Spin 6 column. The non-oxidized and oxidized genomic DNAs were treated with MethylCode bisulfite conversion kit (Invitrogen). Loci-specific PCRs were performed using PyroMark PCR kit (Qiagen). Amplicons were pooled together and barcoded libraries were prepared by TruSeq library preparation kit (Illumina). Amplicon sequencing was performed on MiSeq (Illumina).

Computational Analysis oxBS Sequencing Data

Bisulfite and oxidative bisulfite sequencing data were mapped against mm9 using the Bismark software (PMID 21493656) v0.6.4 (−q−n 2−chunk mbs 1028 bowtie−0.12.7). Subsequently, number of reads containing converted and the number of reads containing unconverted cytosines at covered cytosines were counted based on Bismark's mapping results using custom scripts. For CpGs covered by at least 100 reads in both, the BS and oxBS sample, the percentage of hydroxymethylation has been calculated by subtracting the observed methylation in ox-bisulfite from the observed methylation in bisulfite.

### RNA-Sequencing (RNA-Seq)

˜70,000 HSCs were sorted into Trizol from the pools of each age group. RNA was isolated with the RNeasy Micro column (Qiagen, Valencia, Calif.). Paired end libraries were generated by using Illumina TruSeq RNA sample preparation kit. Illumina HiSeq was used for sequencing with a paired-end sequencing length of 100 bp.

### ChIP-Sequencing (ChIP-seq)

Chromatin Immunoprecipitation (ChIP) was performed as follows: 20,000-50,000 HSCs (SPKLSCD150+) were sorted and crosslinked with 1% formaldehyde at room temperature (RT) for 10 min, and the reaction was stopped by 0.125M glycine at RT for 5 min. Then the cells were washed once with ice cold PBS containing protease inhibitor cocktail (PIC; Roche) and the cell pellet was stored at −80° C. Cross-linked cells were thawed on ice and lysed in 50 μL Lysis buffer (10 mM Tris pH 7.5, 1 mM EDTA, 1% SDS),then diluted with 150 μL of PBS/PIC, and sonicated to 200-500 bp fragments (Bioruptor, Diagenode). The sonicated chromatin was centrifuged at 4° C. for 5 mM at 13,000rpm to remove precipitated SDS. 180 μL was then transferred to a new 0.5 ml collection tube, and 180 μL of 2×RIPA buffer (20 mM Tris pH 7.5, 2 mM EDTA, 2% Triton X-100, 0.2% SDS, 0.2% sodium deoxycholate, 200 mM NaCl/PIC) was added to recovered supernatants. A 1/10 volume (36 μL) was removed for input control. ChIP-qualified antibodies (0.1 μg H3K4me3 Millipore 07-473, 0.3 μg H3K27me3 Millipore 07-449, 0.1 μg H3K79me2 Abcam ab3594) were added to the sonicated chromatin and incubated at 4° C. overnight.

Following this, 10 μL of protein Amagnetic beads (Dynal, Invitrogen) previously washed in RIPA buffer were added and incubated for an additional 2 hours at 4° C. The bead: protein complexes were washed three times with RIPA buffer and twice with TE (10 mM Tris pH 8.0/1 mM EDTA) buffer. Following transfer into new 1.5 ml collection tube, genomic DNA was eluted for 2 hours at 68° C. in 100 μL Complete Elution Buffer (20 mM Tris pH 7.5, 5 mM EDTA, 50 mM NaCl, 1% SDS, 50 μg/ml proteinase K), and combined with a second elution of 100 μL Elution Buffer (20 mM Tris pH 7.5, 5 mM EDTA, 50 mM NaCl) for 10 min at 68° C. ChIPed DNA was purified by MinElute Purification Kit (Qiagen) and eluted in 12 μL elution buffer. ChIPed DNA were successfully made library using ThruPLEX-FD preparation kit without extra amplification (Rubicon, Ann Arbor, Mich.). Sequencing was performed according to the manufacturer's protocol on a HiSeq 2000 (Illumina). Sequenced reads were mapped to the mm9 mouse genome and peaks were identified by model-based analysis of ChIP-seq data (MACS).

### Analysis of Whole Genome Bisulfite Sequencing (WGBS) Data

The WGBS data analyses were based on BSMAP and a newly developed program MOABS: MOdel based Analysis of Bisulfite Sequencing (Sun et al. http://code.google.com/p/moabs/, manuscript in preparation). Four modules, MOABS, mMap, mCall, mOne and mComp, from this software were used. MOABS seamlessly integrates alignment, methylation ratio calling, and identification of hypomethylation for one sample and differential methylation for multiple samples, and other downstream analysis.

### Reads Mapping

BSMAP was used to align the paired-end bisulfite treated reads to the mouse genome mm9. The adaptor and low quality sequences were automatically trimmed by BSMAP. For each read, the mapping location was determined to be the location with the fewest mismatches. If a read can be mapped to multiple locations with the same fewest mismatches, this read is determined as a multi-mapped read and its mapping location was randomly selected from all mapping locations.

### Under-Methylated Regions (UMRs)

Similar to DMR detection, a two-state first order Hidden Markov Model (HMM) was used to detect highly methylated and lowly methylated regions from a single sample. Only locations with coverage more than 10 reads were considered to increase the detection accuracy. Consecutive CpGs with the same hidden “low methylation” state were merged to form a low-methylation region (LMR). A random shuffle of all the CpGs in the genome, followed by the same procedure for LMR detection was also performed. The resulting NULL distribution indicates the number of CpGs required for LMR detection. With false discovery rate (FDR) at 5%, each LMR will include at least 4 CpGs for WT HSC or at least 5 CpGs for Dnmt3a-Knockout HSC. The UMRs are a subset of LMRs with mean methylation ratio less than 10%. Several highly methylated CpGs may separate two neighboring UMRs. Two such UMRs were merged into a single UMR if the mean methylation ratio of the newly merged UMR is still less than 10%. UMRs less than 1 kb long not used in this work. UMRs greater than or equal to 3.5 kb long were defined as “Canyon”. UMRs greater than or equal to 1 kb but less than 3.5 kb are used as control UMRs (cUMRs) to compare with Canyons to show that Canyons are very unique.

### Analysis of 5hmc CMS Pull Down and Histone Modification ChIP-Seq Data

The 5-hydroxy-methylation CMS samples were sequenced at paired-end 100 bp long. The reads were mapped to the mouse genome mm9 using BSMAP by allowing at most 4 mismatches. Only uniquely mapped reads were used for MACS peak calling at p-value cutoff E-5. Peaks are regions with enrichment in CMS pull down sample compared to control sample. The control is sonicated sample followed by bisulfite conversion but without CMS pull down. The common peaks are those that overlap between wild-type and knock-out samples, while the sample specific peaks are those that do not overlap. To quantitatively detect the difference between two samples, all peaks from both samples were merged to form a new set of synthetic peaks, based on which a Poisson test was performed to detect if one sample has more reads than the other sample in each synthetic peak. Before the test, the read number is normalized to 10 million for every sample.

The same pipeline above was used to analyze histone modification ChIP-seq data, with a few exceptions. The histone modification ChIP-Seq reads were mapped to mouse genome mm9 using SOAP2 by allowing at most 2 mismatches for 50 bp long short reads and at most 4 mismatches for 100 bp long short reads. Only uniquely mapped reads are kept. To remove PCR resulted duplicate reads, at most 2 duplicate reads are allowed for each biological replicate. The number 2 is based on Poisson P-value cutoff of 1×10−5 determined by the total number of reads with respect to the theoretical mean coverage across the genome. The uniquely mapped and duplicate removed reads from each biological replicate are fed as treatment file into program MACS, to find the enriched regions, “peaks.” The H3K27me3 peaks are called by SICER with parameters “window size 200 fragment size gap size 600 and FDR 1E-8”. The peaks from all biological replicates of a specific sample are merged to form the final set of peaks for this specific sample.

### Analysis of RNA-Seq Data

Paired-end 100 bp reads were sequenced for RNA-seq. The last 20 bases were trimmed due to average low quality. The alignment was performed by RUM 43, which first mapped reads to the genome and transcriptome by Bowtie, and then used blat to re-map those initially unmapped reads to the genome. The information from the two rounds of mappings was merged. The multiply mapped reads were discarded. The gene annotations used for transcriptome alignment include refSeq, UCSC known Gene and ensemble gene models. The gene expression, FPKM value, was calculated by counting the reads matching the exons of each gene. Differential expression was performed using edge R.

### UMR Dynamics in Size and Methylation Ratio

The UMR dynamics were defined by size including expanded, shrunk and unchanged between wild-type and knock-out samples using the following criterion: if one edge of a wild-type UMR moves outward, or inward in the knockout sample, for more than 200 bases, this edge is classified as “expanded” or “shrunk”, respectively. If the change is less than 200 bases, the edge is classified as “unchanged”. Furthermore, if the wild-type UMR disappears in knock-out sample, both edges of the UMR are classified as “shrunk”; whereas both edges of an emerging new UMR in knock-out sample are classified as “expanded”.

### Analysis of Oncomine-AML Genes

Oncomine (Compendia Biosciences Ann Arbor, Mich. USA) was used to assess the enrichment of Canyon-associated genes expressed in WT murine HSCs (FPKM>1) in patient signatures of genes over-expressed in Leukemic disease vs. normal bone marrow. Oncomine assesses overlap significance with Fisher's exact test. Our threshold criteria were Odds Ratio≧1.8 and p-value<1E-5. To address the challenge of cross-species comparison as well as the inherent technical limitations of comparing next generation sequencing data to that derived from legacy microarray technologies, we limited our analysis to signatures derived from the two most recent 3′IVT Affymetrix expression arrays represented in Oncomine, hgu133a and hgu133plus2, which interrogate 12,624 and 19,574 unique genes, respectively. Generation of random gene sets (each approximating the number of expressed Canyon genes) and mapping of mouse to human gene homologs were performed in R with the Bioconductor package ‘annotation Tools’ (Kuhn 2008). Simulated Canyons Genes represent randomly sampled genes with promoter enrichment or depletion of H3K27me3 histone modifications proportionate to the distributions observed in WT HSC Canyons as determined by ChIP-seq. Random unmethylated promoters were sampled from promoters (excluding Canyon genes) with mean CpG methylation level <10% in WT HSC. Promoters regions were defined as ±1 kb relative to TSS in Refseq transcripts. Random expressed genes were sampled from genes with FPKM>1 in WT HSC.

### DNMT3A Mutant AML Cell Lines

DMNT3A-mutant human AML cell lines OCIAML2 and OCIAML3 were used in these studies. These cell lines were found to have increased total H3K79me compared to DNMT3A-wild type controls, consistent with the increased DOT1L expression in Dnmt3a^{-/-} HSCs.

### Biochemical Assays (Cell Proliferation, Apoptosis, Differentiation Analysis)

Patient AML cell lines were plated in media and treated with the DOT1L inhibitors SYC-522, EPZ004777 or DMSO control was added at the indicated concentrations. Cells counted, washed and re-plated in fresh compound at equal densities every 3-4 days for 12 days. Cell growth and viability were followed by serial cell counts and trypan blue exclusion staining.

For the Annexin V apoptosis assay, 1×10⁶ SYC-522-, EPZ004777- or DMSO-treated cells were washed in PBS, resuspended in Ca/HEPES buffer (10 mM HEPES, pH7.4; 140 mM NaCl; 2.5 mM CaCl2) and incubated with Annexin V-APC (BD Pharmingen, San Jose, Calif.) for 30 min then analyzed by flow cytometry.

Cell differentiation analysis was performed by staining with fluorescently labeled marker for cell differentiation, CD14 followed by flow cytometry. Propidium iodide was added prior to flow cytometry analysis to exclude dead cells.

### Example 2

### Identification of Large Under-Methylated Canyons with Unique Genomic Features

Previous WGBS studies demonstrated that hypomethylated regions are enriched for functional regulatory elements such as promoters and enhancers. Here, a Hidden Markov Model was used to identify under-methylated regions (UMRs) with average proportion of methylation≦10% and required at least 5 CpGs per kb to satisfy the permutation-based FDR 5%. Using these criteria, there are 32,325 UMRs in mouse HSC methylome. Most UMRs are associated with promoters or gene bodies and only 8.3% showed intergenic localization. By inspecting the UMR size distribution, a small portion were exceptionally large was observed, with some of them extending over 25 kb, such as the UMR associated with the Pax6 gene, representing an expanse of unmethylated DNA that is considerably larger than that previously reported. In the genome landscape, these large methylation-depleted regions appear as “canyons” cut into a plateau of high methylation, usually sequestering a single gene. In order to determine whether these large UMRs represented some unique genomic feature, they were required to be at least 3.5 Kb in length (>10 times larger than the typical CGI of ˜300 bp11; and see methods); this revealed 1,104 methylation “Canyons” representing 3.4% of all UMRs. To compare these with typical UMRs, we established a control group of 13,579 UMRs (cUMRs) that were longer than 1 kb but smaller than 3.5 kb. This control group eliminates the smallest UMRs that tend to be transcription factor binding sites. To gain insights into the biological function of these Canyons, gene ontology enrichment analysis was performed with Canyon-associated genes and cUMR-associated genes. Canyon-associated genes showed a striking pattern of enrichment for genes involved in transcriptional regulation (318 genes, P=6.2×10-123), as well as genes containing a homeobox domain (111 genes, P=3.9×10-85), comprising one of the most ancient gene families involved in embryonic development of bilaterians. In contrast, the genes associated with cUMRs all give non-significant p-values on these 4 GO terms. Among the largest 20 Canyons, 15 harbor homeobox-containing genes. These Canyons typically extend well outside of the immediate coding regions of these gene. As a group, these Canyons are particularly highly conserved and are depleted for transposable elements and repeats.

### Example 3

### Expression of Canyon Genes is Regulated by Histone Modifications

Low DNA methylation is usually associated with active gene expression. However, many Canyon-associated genes are developmental regulators that are not known to play roles across many cell and tissue types thus we examined their regulatory features in more detail in the hematopoietic system. RNA-seq data indicated that among the twenty largest Canyons, only two harbored highly expressed genes: Hoxa9 and Meis1, which encode transcription factors critical for hematopoiesis and frequently deregulated in leukemia.

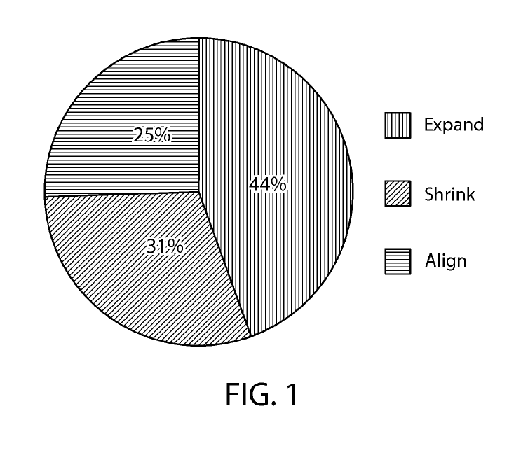

Because DNMT3A is mutated in a high frequency of human leukemias23, the impact of loss of Dnmt3a on Canyon size was examined. All UMRs in HSCs conditionally inactivated for Dnmt3a (KO) were compared to wild-type (WT) HSCs. Upon knockout of DMNT3A, the edges of the cUMRs and Canyons are hotspots of differential methylation while regions inside of cUMRs and Canyon are relatively resistant. Thirty percent of all differentially methylated regions (DMRs) in the Dnmt3a KO were located at the edges of UMRs. This focused methylation loss at the edges of UMRs suggests that Dnmt3 abnormally acts to maintain methylation at their boundaries. On 44% of Canyons, the edges were eroded such that they increased in size, and 31% of Canyons experienced hypermethylation at the edges, such they decreased in size (25% experienced no significant change) (FIG. 1). The methylation loss in Dnmt3a KO HSCs led to the addition of 861 new Canyons for a total of 1787 Canyons. Methylation in some regions that featured a cluster of Canyons in WT HSCs was decimated such that Canyons merged to become groups of larger Canyons (“Grand Canyons”), as exemplified by the HoxB region, in which the enlarged Canyon covers more than 50 kb, interrupted by short stretches of higher methylation. The expansion and contraction of different Canyons in absence of Dnmt3a is reminiscent of the concomitant hyper- and hypo-methylation that is observed in many malignant cells; thus, it should be considered whether other epigenetic mechanisms influenced Canyon behavior. The histone mark distribution on expanding vs. contracting Canyons was thus examined. The H3K27me3 canyons were more likely to contract. This suggests Dnmt3a specifically is acting to restrain Canyon size where active histone marks (and active transcription) are already present.

### Example 4

### Methylation Canyon Gene Expression is Associated with Cancer

Aberrant hyper-methylation in transformed cells has been thought to contribute to malignancy development, and both hyper- and hypo-methylation is associated with transformed cells. Thus, it was tested whether Canyon-associated genes were likely to be associated with hematologic malignancy development. Oncomine was used to assess whether Canyon genes expressed in WT HSCs were associated with the aberrant expression signatures of human leukemias. These Canyon genes were highly enriched in seven signatures of genes over-expressed in Leukemia patients compared to normal bone marrow; in contrast, four sets of control genes were not similarly enriched.

Further, TCGA data was used to test whether Canyon gene expression changes were associated with DNMT3A mutation in AML patients. Remarkably, it was found that expressed canyon genes are significantly enriched for differentially expressed genes between patients with and without DNMT3A mutation (p value<0.05). Overall, 76 expressed canyon genes, including multiple HOX genes, are significantly changed in patients with DNMT3A mutation (p=0.0031). Notably, the previous gene expression comparison in whole transcriptome level did not identify any expression cluster associated with DNMT3A mutation, but here two strong clusters from unsupervised clustering with 80% of Dnmt3a mutant patients enriched into cluster A were identified. The expressed canyon genes identified here may be used as a unique gene expression signature to define the DNMT3A mutation status in patients. Expression of Canyon genes in various other cancer types was analyzed by using data from cancer cell line encyclopedia (CCDE; a compilation of gene expression data from 947 human cancer cell lines). Canyons expressed in HSC are highly expressed or depleted in hematologic cancer cell lines, whereas unexpressed canyons showed high expression in other cancer cell lines, which may reflect the original tissue specificity of canyon expressions which regulated by histone modification.

Furthermore, in analyses of gene expression data it was observed that murine DMNT3A^{-/-} HSCs markedly overexpress the histone 3, lysine 79 (H3K79) methyltransferase, DOT1L. This is of interest given the known functional interplay between DNA methylation and histone modifications. Additionally, DOT1L plays a critical role in leukemia with MLL-rearrangements, lesions that essentially never occur concomitantly with DNMT3A mutations in AML. The mutual exclusion of these lesions combined with the observed overexpression of DOT1L in a murine model, led to the postulation that MLL-rearrangements and DNMT3A mutations are distinct epigenetic aberrations that converge on a common mechanism resulting in dysregulated gene expression, specifically mediated by H3K79 methylation (H3K79me).

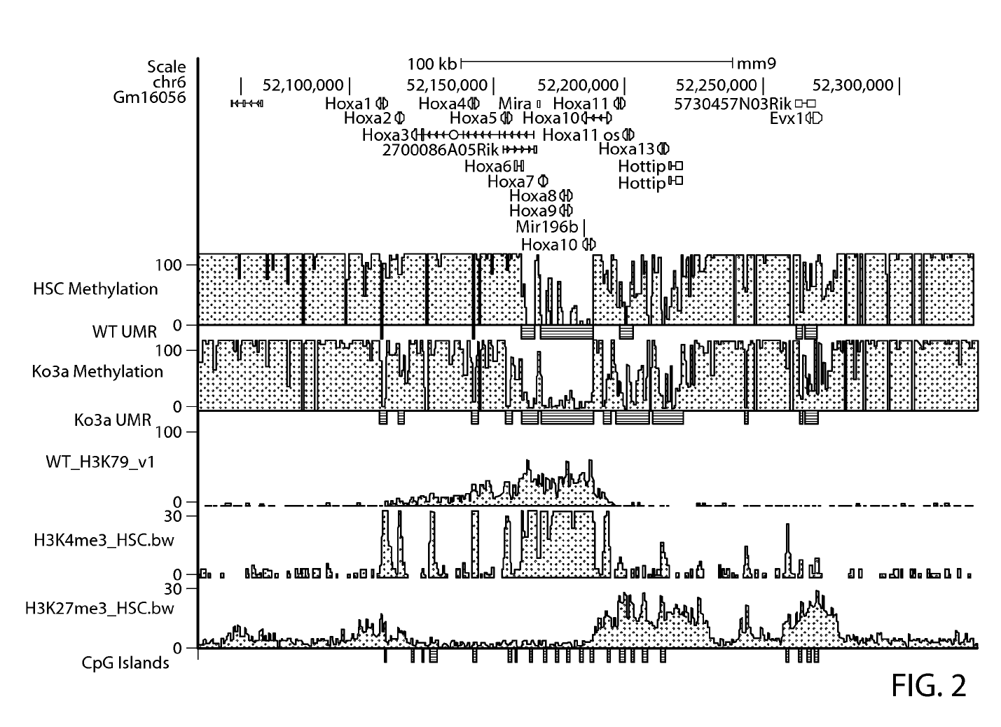

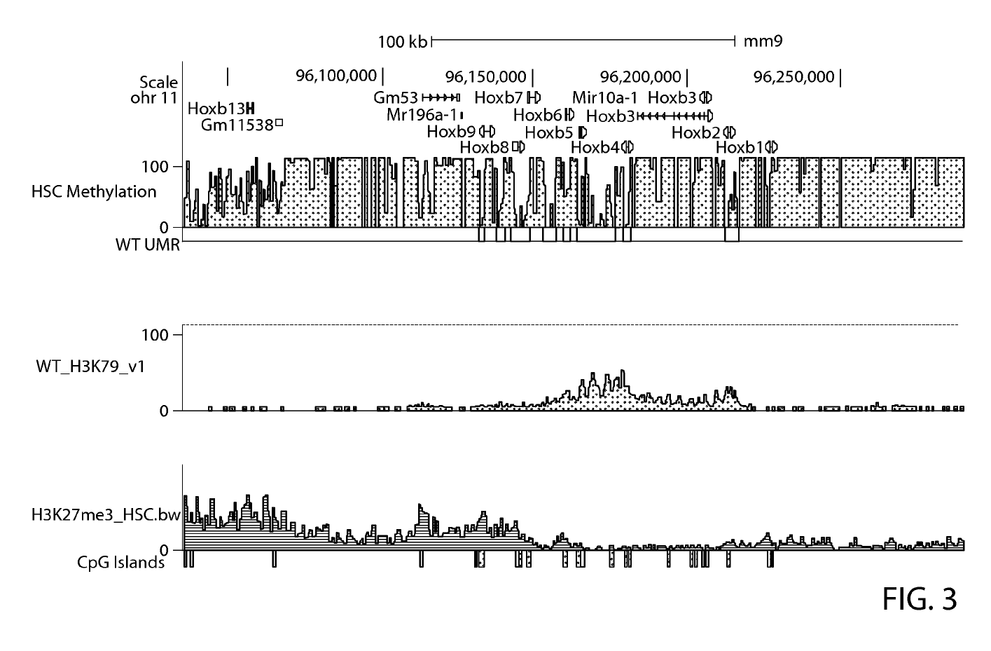

Analysis of TCGA data has confirmed many of these sites as regions with methylation loss in human DNMT3A-mutant AML. Further, many canyon-associated genes, including HOX genes are significantly changed in human DNMT3A-mutant AML. To determine if in DNMT3A-mediated malignant hematopoiesis, H3K79 methylation correlates with altered DNA methylation ChIP-seq for H3K79me2 was performed and the data aligned with whole genome DNA methylation data. This revealed that H3K79me2 specifically coats canyons that expand with DNMT3A loss, including the HoxA and HoxB clusters (FIG. 2 and FIG. 3), This strong correlation between H3K79 methylation and DNA hypomethylation with DNMT3A loss suggests a functional interaction.

### Example 5

### DNMT3A-Mutant AML is Sensitive to DOT1L Inhibition

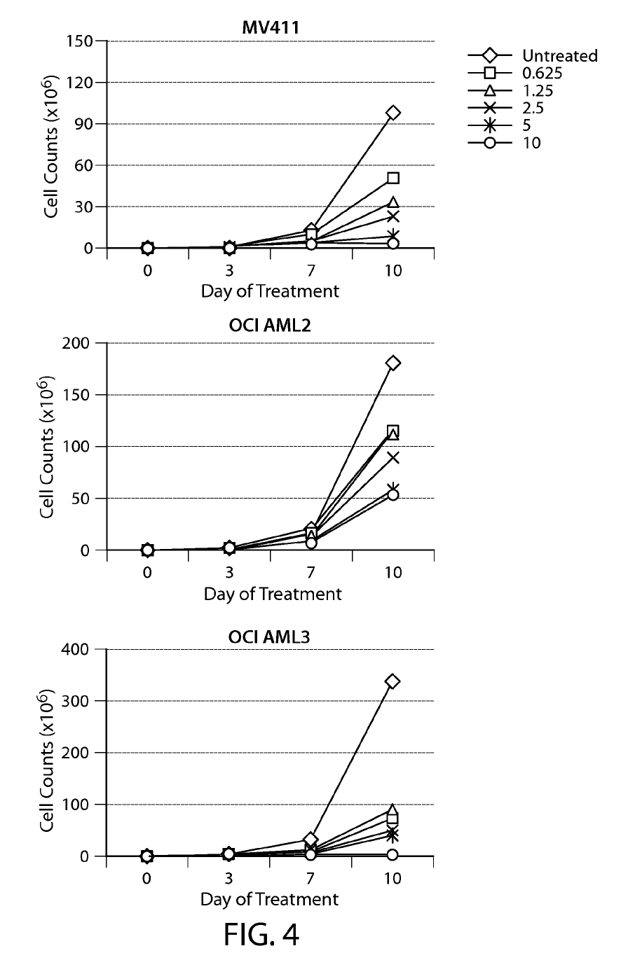

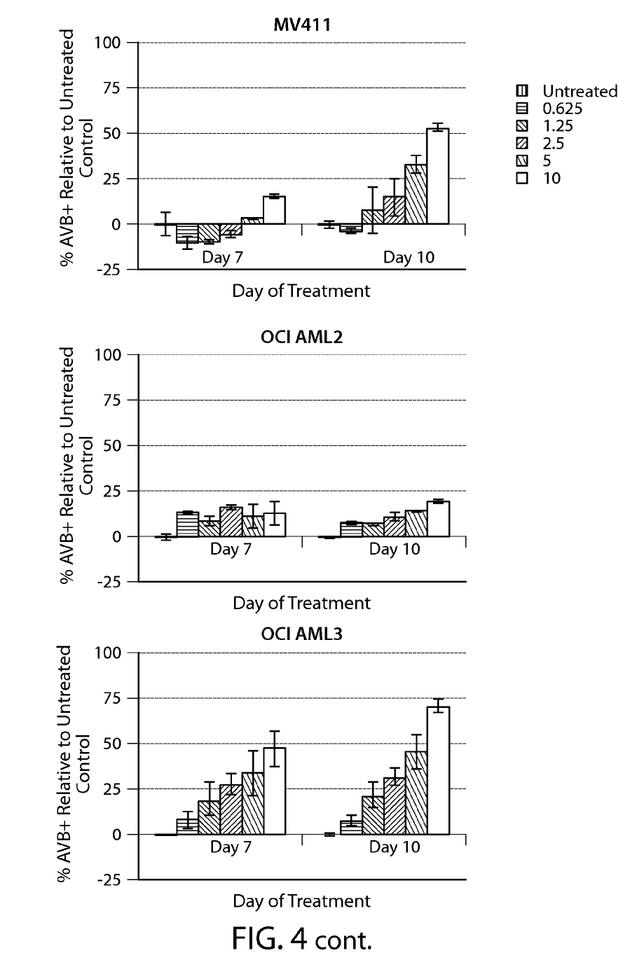

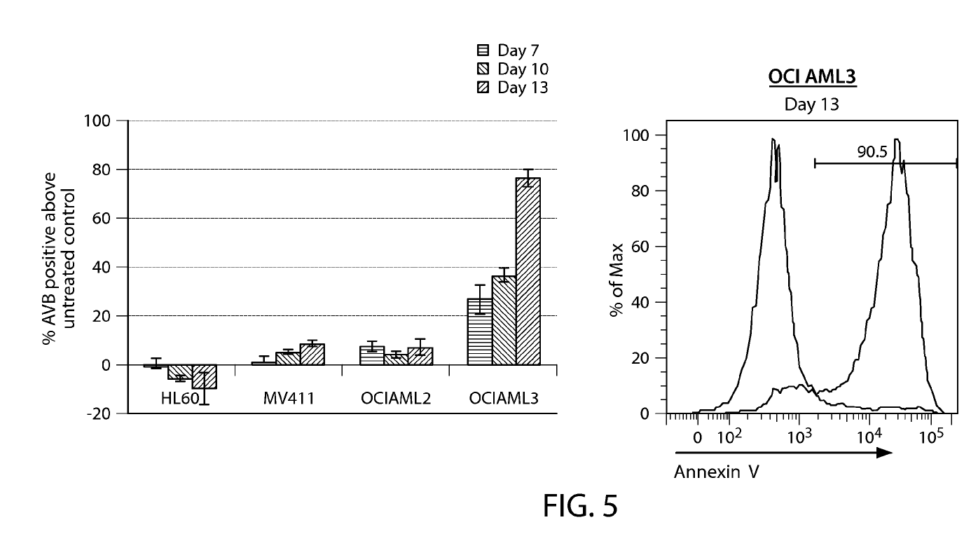

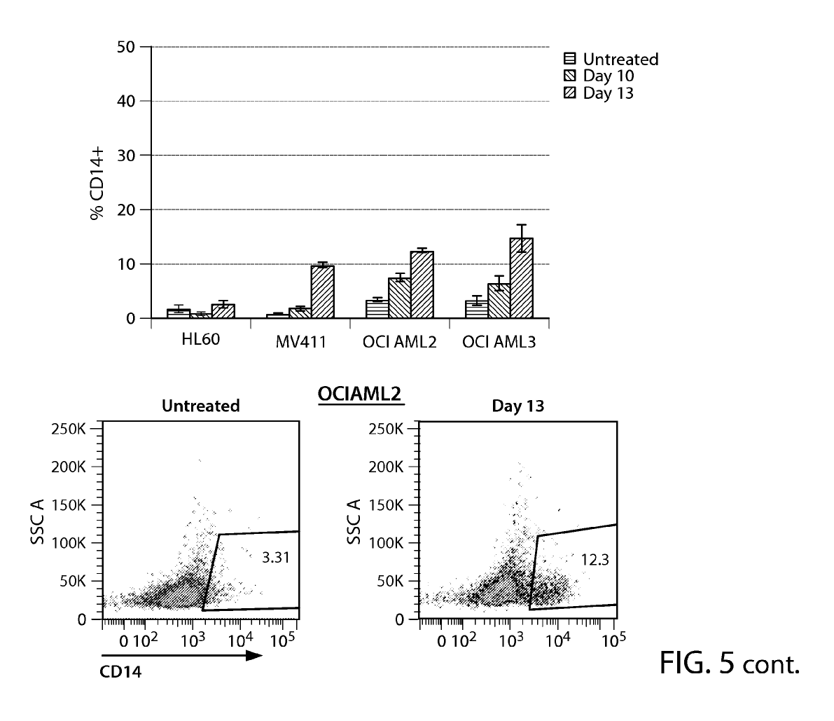

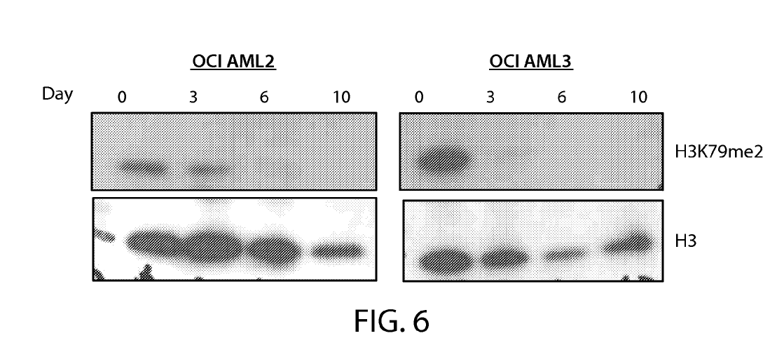

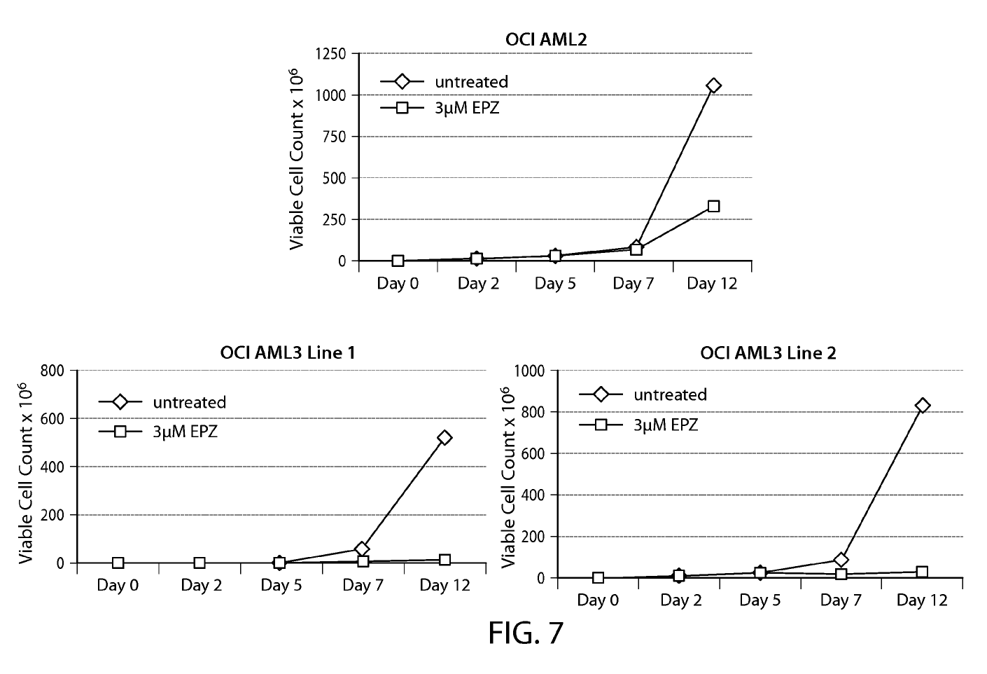

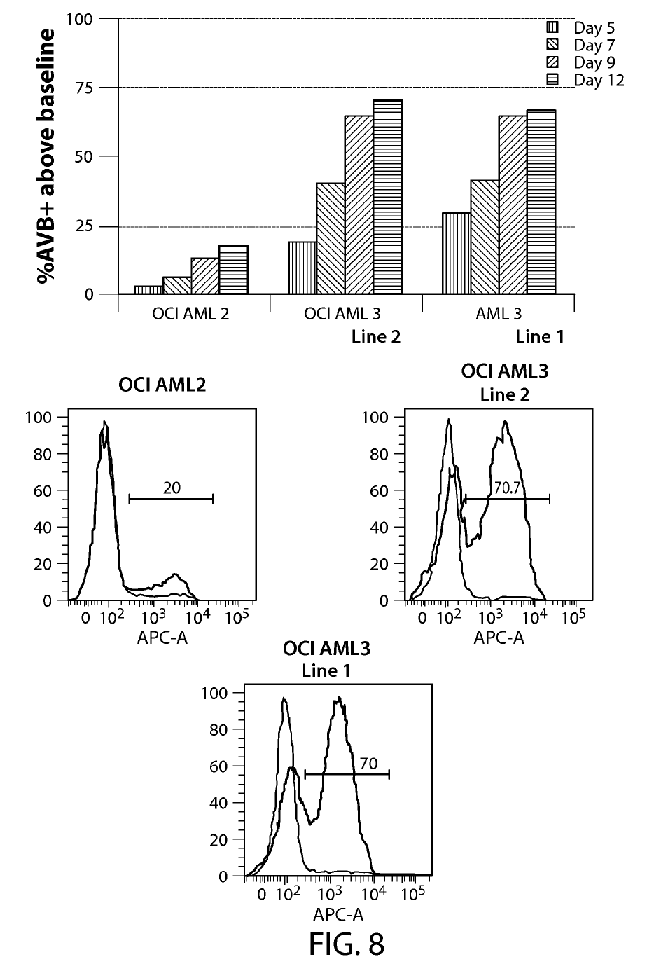

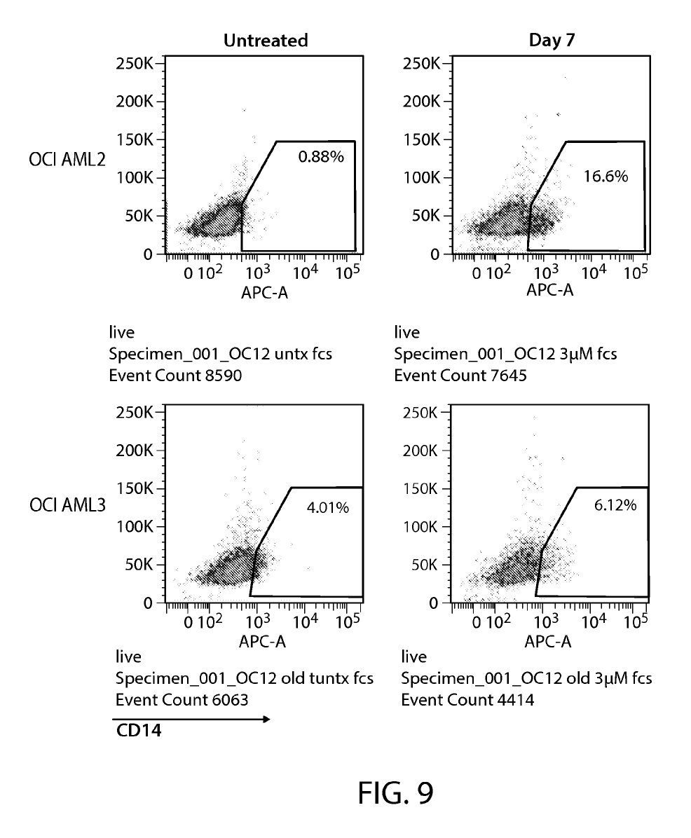

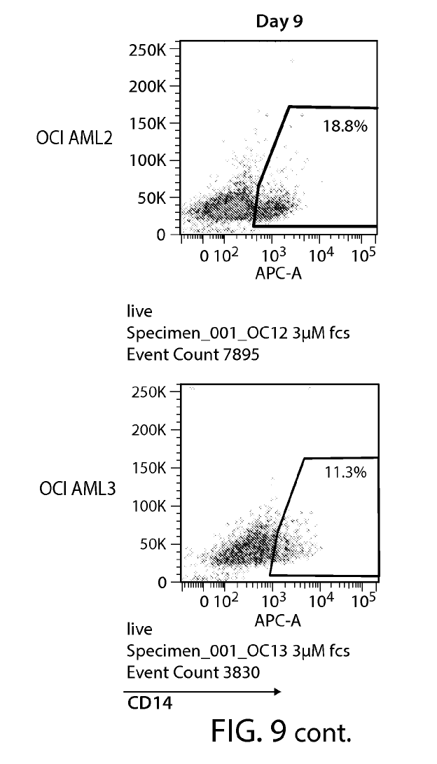

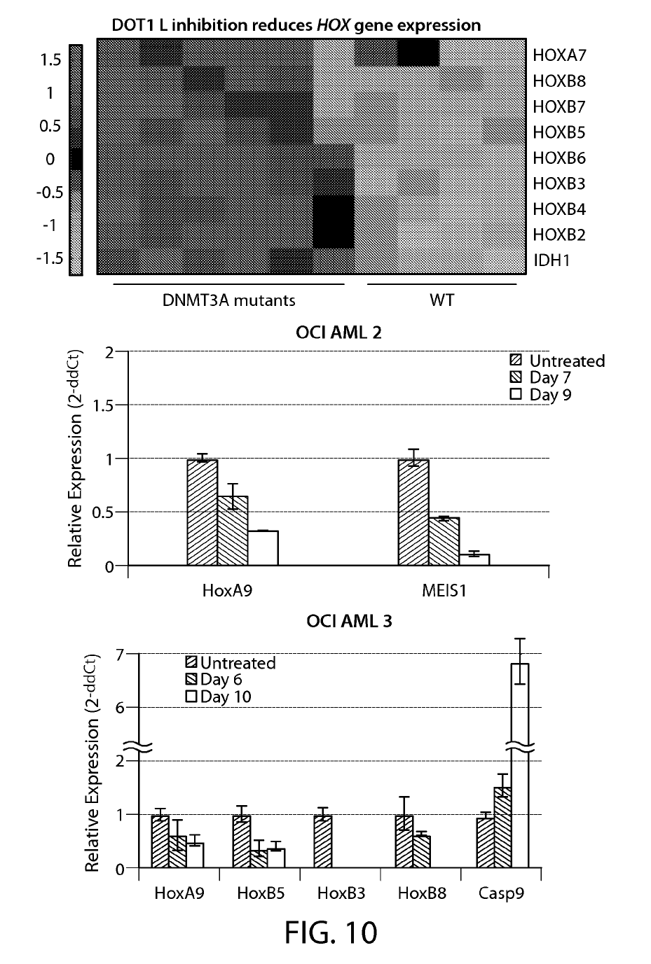

To explore the role of DOT1L in human DNMT3A-mutant AML, the DNMT3A mutant human AML cell lines OCIAML2 and OCIAML3 were studied. These cell lines were found to have increased total H3K79me compared to DNMT3A-wild type controls, consistent with the increased DOT1L expression in DNMT3A^{-/-} HSCs. The in vitro efficacy of the selective DOT1L inhibitors SYC-522 and EPZ004777, was tested against the DNMT3A-mutant cells. Treatment with SYC-522 caused decreased proliferation, induction of apoptosis and terminal differentiation of AML cell lines in a dose dependent manner (FIG. 4 and FIG. 5). SYC-522 was also found to inhibit H3K79 methylation in a time-dependent manner (FIG. 6). Treatment with 3 μM of EPZ004777 also led to a dose- and time-dependent inhibition of proliferation (FIG. 7) and induction of apoptosis (FIG. 8) in the DNMT3A-mutant cell lines at concentrations comparable to those used for MLL-rearranged cell lines. With 3 μM of EPZ004777 treatment, DNMT3A-mutant cells also had evidence of induction of differentiation with increased expression of the mature monocyte marker, CD14 (FIG. 9). Importantly, oncogenic HOX genes overexpressed in DNMT3A-mutant AML were repressed in a time-dependent fashion with SYC-522 treatment (FIG. 10). In conclusion, the data suggest that DOT1L may be a novel, immediately actionable therapeutic target for the treatment of DNMT3A-mutant AML.

### Example 6

### DOT1L as a Therapeutic Target for the Treatment of DNMT3A-Mutant Acute Myeloid Leukemia

### Murine Model:

Dnmt3a-KO in C57B1/6 CD45.2 Dnmt3a^{fl/fl}-Tamoxifen-cre mice was induced by 5 daily intraperitoneal injections of tamoxifen. As controls, Dnmt3a^{WT/WT}-Tamoxifen-cre mice were treated in the same fashion. Eight weeks after tamoxifen induction, bone marrow was harvested for transplantation. Harvested cells (1×10⁶ per mouse) were transplanted by retro-orbital injection into lethally irradiated syngeneic CD45.1 recipients.

### ChIPseq

Four months after transplantation, recipient mice were sacrificed and pooled bone marrow hematopoietic stem cells (HSCs) from Dnmt3aKO and control mice were purified using c-Kit magnetic enrichment (AutoMACS; Miltenyi Biotec) followed by sorting for propidium iodide⁻, lineage⁻, Sca1⁺, CD48, and CD150⁺ cells (FACSAria; BD Biosciences; antibodies from Becton Dickinson). ChIPseq was performed on the purified Dnmt3aKO and control HSCs by cross-linking the chromatin with 1% formalin then lysing the cells in SDS buffer. DNA was fragmented by sonication and chromatin immunoprecipitation performed using a ChIP-grade anti-H3K79me2 antibody (ab3594; Abcam). The eluted DNA was used to prepare a DNA library utilizing the Illumina ChIPseq kit, and then sequenced on an Illumina HiSeq instrument, using 100-base paired-end sequencing.

### Cell Culture

MV411 (CRL-9591) were obtained from ATCC and HL-60 (CCL-240), THP1, Kasumi-1, and KG-1 cells. Cells were grown in RPMI-1640 (Invitrogen) plus 10% FBS, 1% L-glutamine, and 1% pen strep, at 37° C. in 5% CO₂. Cell lines were validated by the short tandem repeat method.

### Cell Proliferation and Viability Assays

Exponentially growing cells were plated, in triplicate, in 24-well plates at a density of 2×10⁵/mL in a final volume of 1 mL. For dose dependent assays, cells were incubated in increasing concentrations of SYC522 up to 10 μM or EPZ004777 up to 24 μM or DMSO control. For time-dependent assays, cells were incubated in 3 μM EPZ004777 or DMSO control. Every 2-3 days media and compound was replaced and cells were split back to a density of 2×10⁵/mL. At each re-plating viable cells number was determined by trypan blue exclusion. Total cell number is expressed as split-adjusted viable cells per mL.

### Flow Cytometry Analysis of Apoptosis, Cell Cycle and Differentiation

Cells were harvested at each re-plating of the cell proliferation assay described above then split for simultaneous analysis of apoptosis, cell cycle, and differentiation. Apoptosis was determined by Annexin V binding (AVB) flow cytometry assay. The harvested cells were re-suspended in 1×AVB buffer, stained with APC-conjugated Annexin V (BD), then analyzed by flow cytometry. For cell cycle analysis, cells were pelleted, washed in PBS with 0.5% bovine serum albumin, then fixed in 70% ice-cold methanol. The fixed cells were then incubated in PBS with 100 μg/mL RNAse (Invitrogen) for 15 minutes in a 37° C. water bath. Propidium iodide (PI) in PBS at a final concentration of 25 μg/mL was then added and the cells were analyzed by flow cytometry. For differentiation, cells were pelleted, and stained with APC-conjugated CD14 then washed and re-suspended in media containing PI to exclude dead cells. All analyses were performed using an LSR II flow cytometer (BD biosciences) and the data were analyzed using FlowJo (TreeStar, V8.8.7).

### Quantitative Real-Time PCR

RNA was extracted from OCI AML2 and OCI AML3 cells treated with 3 μM EPZ004777 or DMSO control using RNeasy RNA isolation kit (Qiagen) per manufacturer's protocol. RNA was quantified using Nanodrop instrument and 300-1000 ng (equal quantities for EPZ004777 treated and DMSO controls) of RNA were reverse transcribed using the SuperScript® III First-Strand Synthesis System (Invitrogen). Reverse transcription quantitative PCR was then performed in triplicate using predesigned TaqMan primer and probe sets for HOXA9 (Hs00365956_m1), MEIS1 (Hs01017441_m1), HOXB8 (Hs00256885_m1), HOXB3 (Hs01587922_m1), and GAPDH (Hs02758991_ml) (FAM-MGB; Applied Biosystems) for 30 cycles using an AbiPrism 7900HT (Applied Biosystems). Target gene numbers were normalized to the ribosomal RNA 18s (VIC-MGB; Applied Biosystems) and fold-change determined by the 2^{-ΔΔCT} equation.

### Colony-Forming Cell Assays

Viably frozen human primary AML samples were thawed quickly and placed in fresh RPMI 1640 (Invitrogen) plus 10% FBS, 1% L-glutamine, and 1% pen strep. After recovery for 2-3 hours in media, viable cells were counted by trypan blue exclusion and plated in triplicate (5,000 viable cells/plate) in human H4034 methylcellulose media (StemCell Technologies) with 3 μM EPZ00477 or DMSO vehicle control. After 12-14 days, plates were scored for colony counts and colony morphology. Cells were then isolated from the plates, stained for CD45, CD14, CD13 and analyzed by flow cytometry. Cell morphology was examined by H&E staining of cytospins of isolated cells prepared using the CytoPro cytocentrifuge.

### Nude Rat OCI-AML3 Xenograft Study

In vivo studies were conducted. Human acute myeloid leukemia cells OCI-AML3 were implanted subcutaneously into the right flank of female athymic nude rats (Hsd:RH-Foxn1rnu, Harlan Laboratories, Inc.). EPZ-5676 was delivered by continuous IV infusion via a catheter surgically implanted in the femoral vein of each rat. Animals were separated into either an efficacy or PK/PD cohort. Both cohorts were dosed by continuous IV infusion with 35 or 70 mg/kg/day. A control group received continuous IV infusion of the vehicle, 5% hydroxypropyl-β-cyclodextrin (HPBCD) in saline. Efficacy was determined after 21 days of drug treatment followed by a 7 day drug holiday. Animals assessed for PK/PD were dosed for 14 days and euthanized following the completion of infusion. All rats were weighed and tumors calipered twice weekly until the end of study. At the completion of the study animals were euthanized by terminal cardiac puncture under isoflurane anesthesia. Euthanized rats were sampled for tumor tissue. Tumors were collected in an RNase-free environment, bisected, snap frozen in liquid nitrogen, pulverized and finally stored at (−80° C.).

### Histone Extraction

For isolation of histones from bone marrow, cells were lysed in in 250 μL nuclear extraction buffer (10 mM Tris-HCl, 10 mM MgCl2, 25 mM KCl, 1% Triton X-100, 8.6% Sucrose, plus a Roche protease inhibitor tablet 1836145). The samples were incubated on ice for 5 minutes, then nuclei were collected by centrifugation at 600 g for 5 minutes at 4° C. and washed once in ice cold PBS. Supernatant was removed and histones were extracted for one hour with 0.4 N ice cold sulfuric acid. Extracts were clarified by centrifugation at 10,000 g for 10 minutes at 4° C. and transferred to a fresh microcentrifuge tube containing 10×volume ice cold acetone. Histones were precipitated at −20° C. for 2 hours, pelleted by centrifugation at 10,000 g for 10 minutes, and resuspended in 150 μL water. Histones were quantified using the BCA protein assay (Pierce, 23225).

For isolation of histones from ground tumor powder, the above protocol was performed with the following exceptions. Approximately 20 mg of tumor powder was lysed in 500 μL nuclear extraction buffer. A 5 mm steel bead was added to each sample (Qiagen, 69989) and the samples were lysed on the TissueLyser (Qiagen, 85210) for 30 seconds at 30 /s frequency. Sample blocks were rotated 180° and lysed for another 30 seconds at 30 /s frequency.

### ELISA Analysis on Inhibition of H3K79me2

ELISA was performed. Briefly, histones were run in matched H3K79me2 and total H3 ELISAs to calculate total levels of H3K79 methylation and total histone H3, respectively. The optical density of the H3K79me2 ELISA was normalized to that of the total H3 ELISA for each sample.

### Quantitative Real-Time PCR

For the isolation of RNA, 10 mg of tumor powder was lysed in 600 μL RLT Lysis Buffer (Qiagen) and homogenized with a Qiashredder column (Qiagen, 79656) following the manufacturer's protocol. Flow-through was collected and total RNA was isolated using the RNeasy Total RNA isolation kit (Qiagen, 74106) according to manufacturer's instructions. MEIS1 and HOXB3 mRNA levels were assessed and normalized to GAPDH by qRT-PCR.

### Dnmt3^{-/-} HSCs are Characterized by Increased DOT1L Expression and Increased H3K79 Methylation

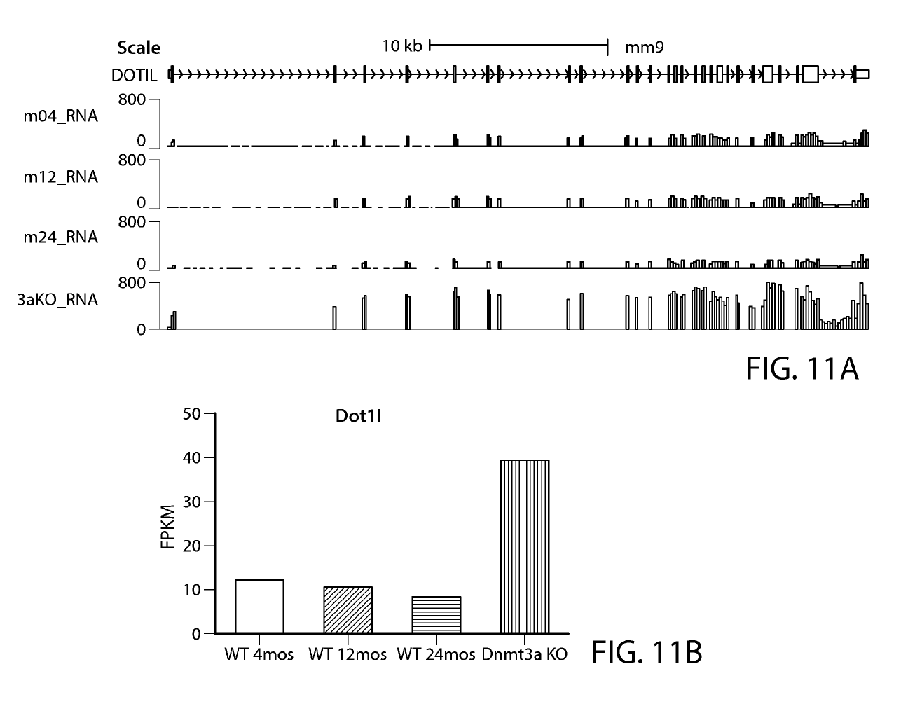

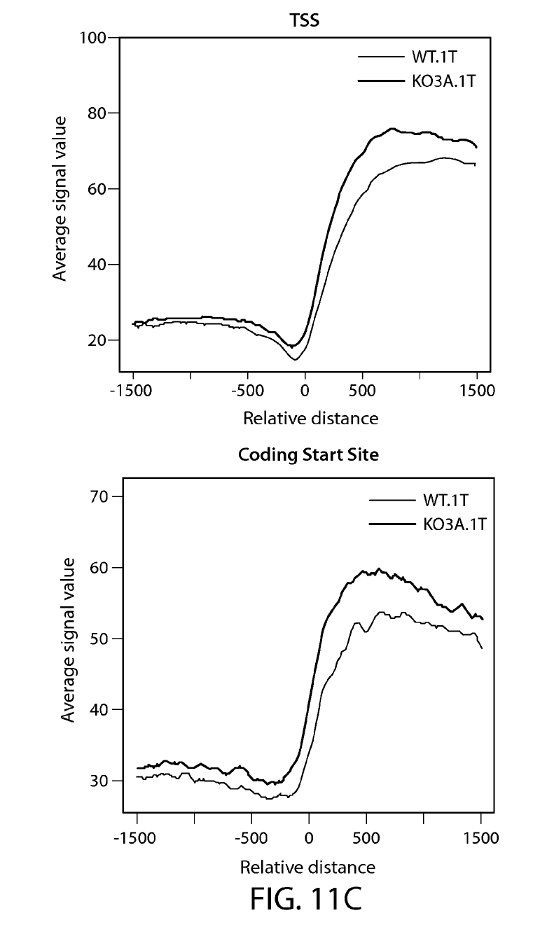

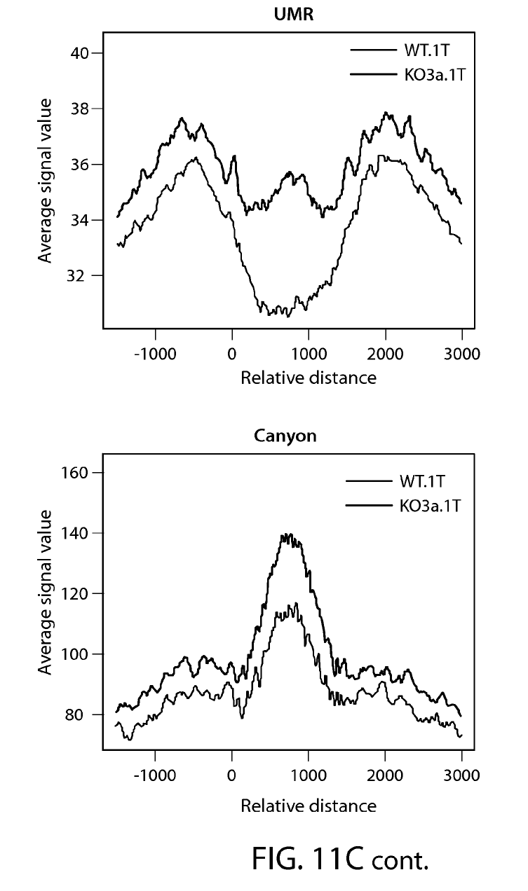

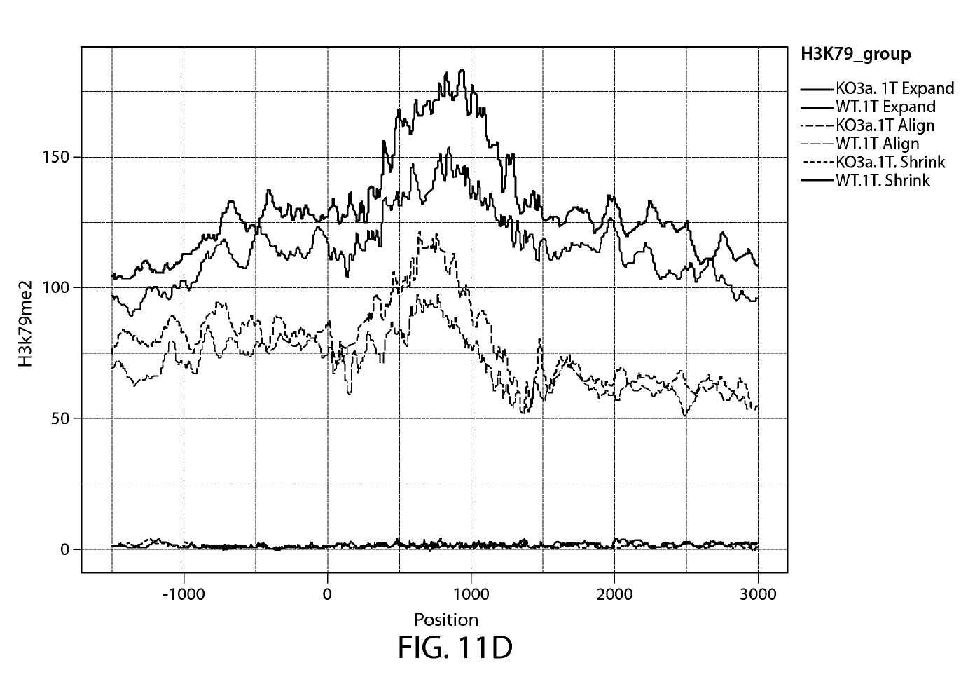

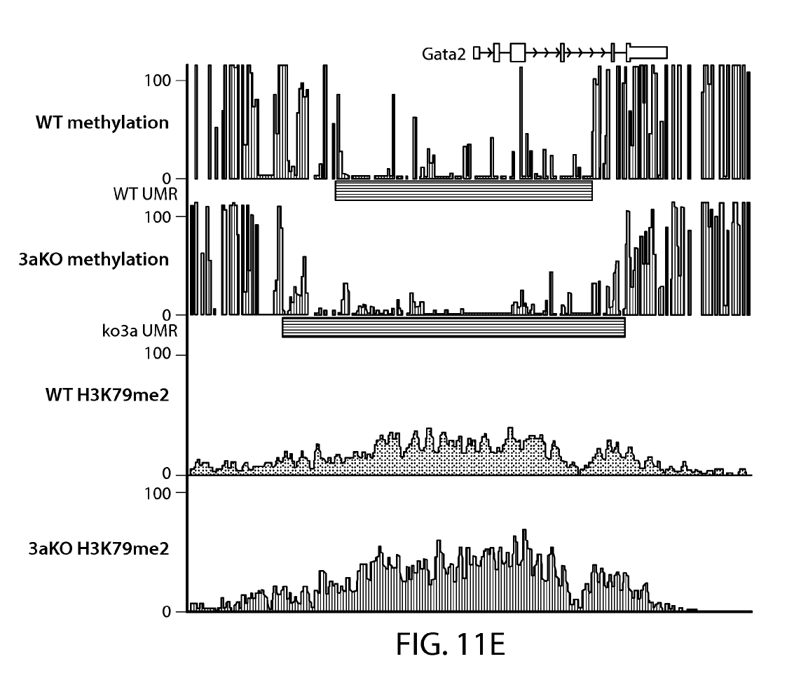

RNAseq performed on Dnmt3a^{-/-} HSCs revealed that DOT1L was overexpressed relative to wild type HSCs (FIG. 11A). Whether if DOT1L-induced H3K79 dimethylation (me2) was also altered in Dnmt3a^{-/-} HSCs was investigated. A close association between canyon methylation changes and the associated histone marks is reported. Expanding canyons typically are coated by the activating H3K4 tri-methyl (me3) mark and lack the repressive histone mark H3K27me3 suggesting that Dnmt3a is particularly important in maintaining DNA methylation specifically at canyons with activating histone marks and active gene transcription. H3K79me may be another key component of this activating histone signature. To investigate if DOT1L-induced H3K79me correlates with altered DNA methylation in Dnmt3a-mediated malignant hematopoiesis, chromatin immuno-precipitation followed by high throughput DNA sequencing (ChIPseq) for H3K79me2 was performed. These data were aligned with whole genome DNA methylation data from hematopoietic stem cells from our Dnmt3a^{-/-} murine model. With Dnmt3a loss, the density of H3K79me2 was significantly increased at transcription start sites, protein coding start sites, and at undermethylated regions including a substantial increase specifically at DNA methylation canyons (FIG. 11B). The association between H3K79me2 and canyon behavior with Dnmt3a loss was then investigated. H3K79me2 is essentially absent at canyons that gain DNA methylations with Dnmt3a loss, is present at relatively low amounts in canyons that align with Dnmt3a loss, and densely coats those canyons that expand with Dnmt3a loss. (FIG. 11C and FIG. 11D). FIG. 11E shows a representative DNA methylation canyon that expands with Dnmt3a deletion. This strong correlation between H3K79me and DNA hypomethylation with Dnmt3a loss suggests a functional interaction.

### DOT1L-Induced H3K79 Methylation in DNMT3A-mutant AML

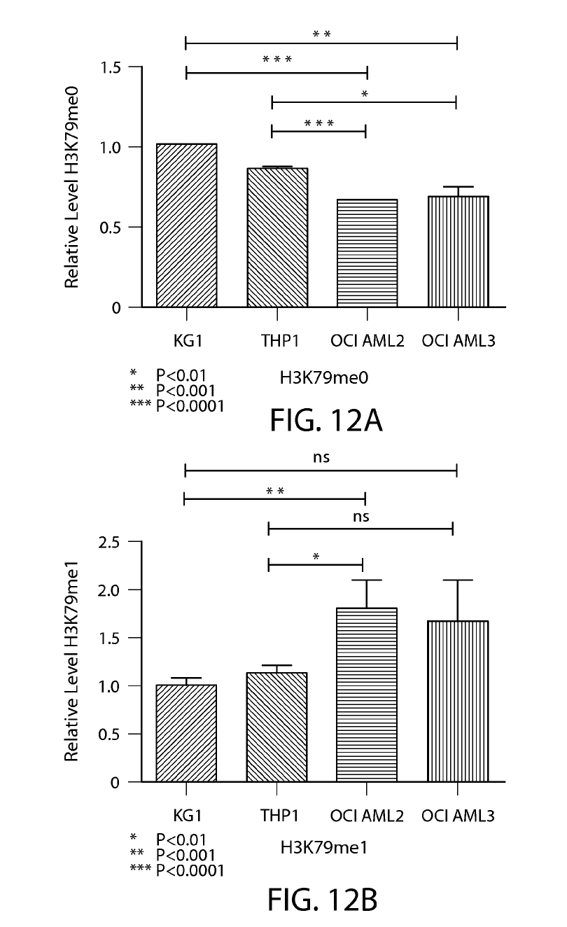

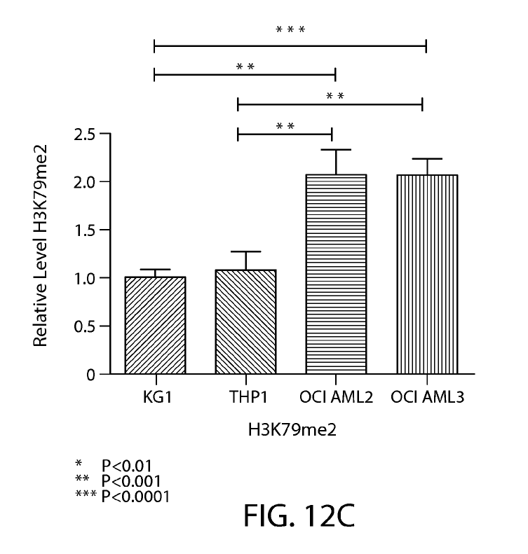

To explore the role of DOT1L in DNMT3A-mutant human AML, DNMT3A-mutant human cell lines OCI AML3 which harbor the most common and well-characterized type of DNMT3A mutation, the dominant negative acting R882 mutation and OCI AML2 cells which have a non-R882 mutation that is functionally uncharacterized, were used. As controls, MLLr cell lines, THP1 and MV411, and KG-1, Kasumi-1, and HL60 cells that have wild type DNMT3A and MLL were used. The relative methylation of H3K79 was determined by mass spectroscopy, which demonstrated decreased unmethylated H3K79 (FIG. 12A), increased H3K79me1 (FIG. 12B) and H3K79me2 (FIG. 12C) in DNMT3A-mutant cells compared to wild type cells, consistent with the overexpression of DOT1L and increase H3K79me density observed in the murine Dnmt3a^{-/-} model (FIG. 12A).

### Pharmacologic DOT1L Inhibition Reduces Cellular H3K79me and Oncogenic HOX Gene Expression in DNMT3A Mutant AML Cells

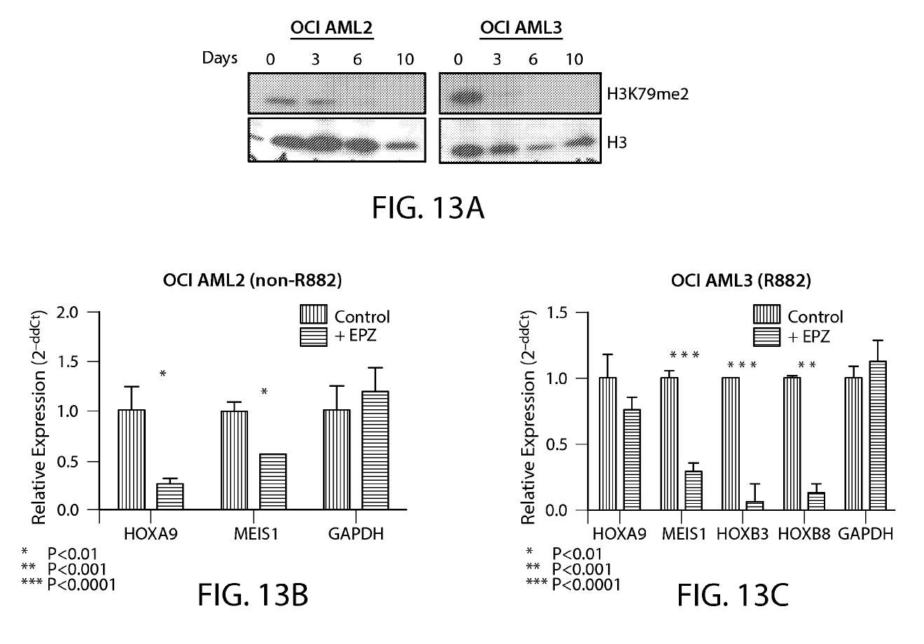

The efficacy of pharmacologic DOT1L inhibition was tested in vitro using two specific DOT1L inhibitors; SYC-522, and EPZ004777. These structurally similar compounds both work by binding the DOT1L-nucleosome complex and competitively inhibiting the binding of the methyl donor, S-adenosylmethionine, and have comparable potency and specificity. DNMT3A-mutant human AML cell lines OCI AML2 and OCI AML3 were treated with SYC-522 or EPZ004777. After treatment, H3K79 methylation was inhibited in a dose- and time-dependent fashion (FIG. 13A). DOT1L inhibition of the expression of leukemogenic genes was then tested. HOXA and HOXB cluster genes were examined as analysis of TCGA data revealed several genes as overexpressed in DNMT3A-mutant patients compared to DNMT3A-wild type patients. Changes in the expression of MEIS1, a gene that is highly expressed in most DNMT3A-mutant AML samples was also examined, because decreased expression of this gene correlates to responses to DOT1L inhibition in MLL-rearranged leukemia. These were also genes of interest, as each are associated with a H3K79me2-coated DNA methylation canyons in murine Dnmt3a^{-/-} HSCs. Treatment with 3 μM of EPZ004777, reduced the expression of HOXA9 and MEIS1 in the OCI AML2 cells (FIG. 13B) and the expression of MEIS1 and predominantly HOXB cluster genes in the OCI AML3 cells (FIG. 13C). The expression of GAPDH was not affected by DOT1L inhibitor treatment, indicating this decreased expression was not due to a general inhibitory effect on gene expression. This suggests that reduction of H3K79me by pharmacologic inhibition of DOT1L can reverse the oncogenic gene expression program of DNMT3A-mutant AML.

Treatment with Pharmacologic Inhibitors of DOT1L Inhibits the Cellular Proliferation of DNMT3A Mutant AML Cell Lines in a Dose- and Time-Dependent Fashion

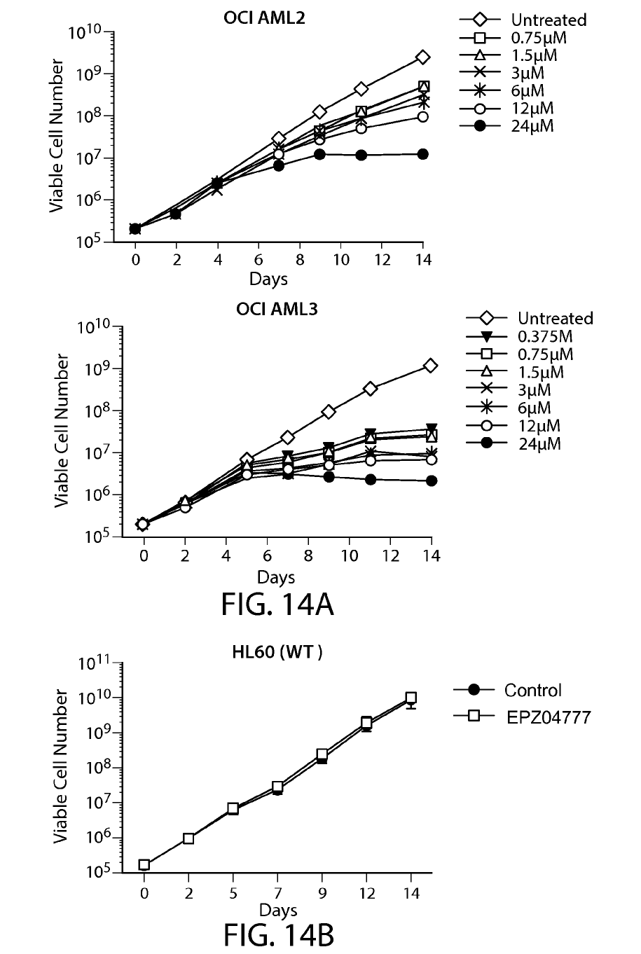

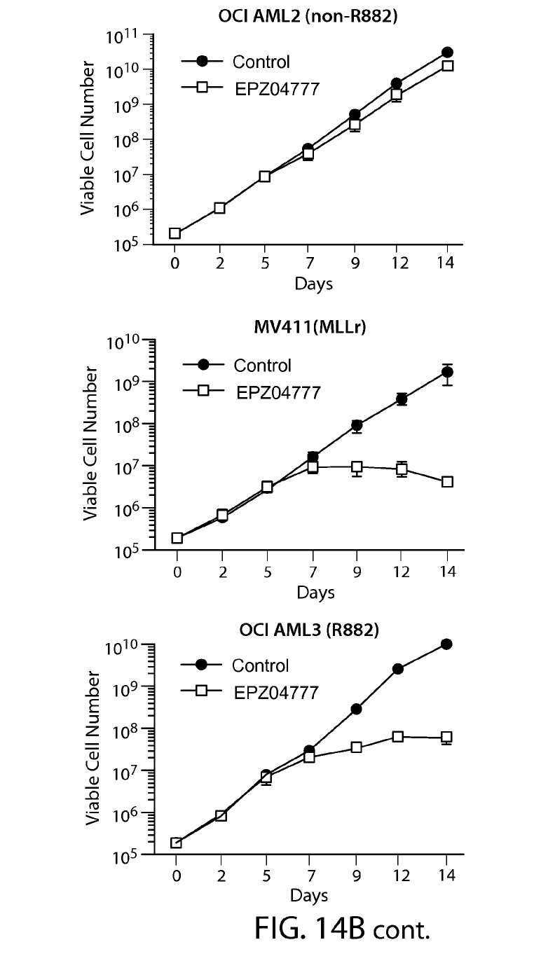

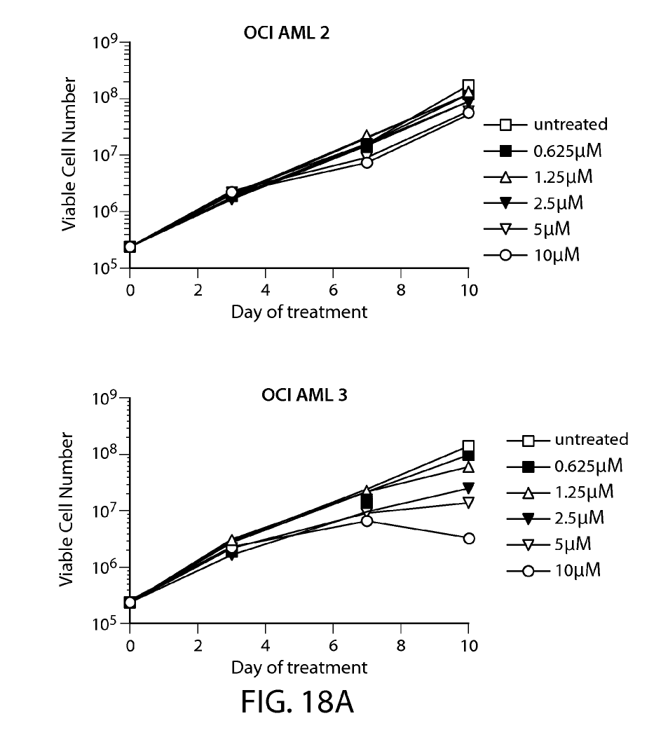

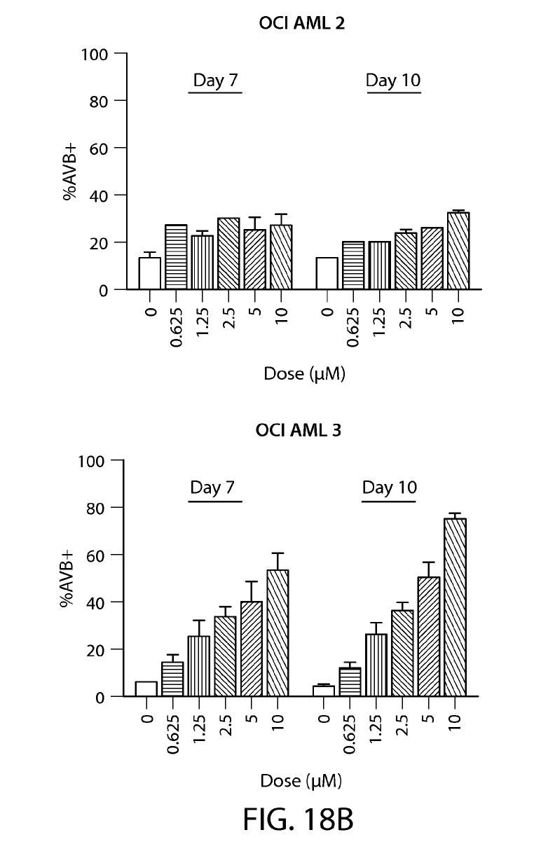

The impact of DOT1L inhibition on cellular proliferation was examined. For all assays, cells were treated with SYC-522 or EPZ004777. Cells were counted and re-plated at a constant cell concentration in fresh drug- or vehicle- containing media every 2-3 days. DNMT3A mutant and OCI AML2 and OCI AML3 cells were first treated in escalating doses of both SYC-522 and EPZ004777 for 10-14 days. Both compounds inhibited growth in a dose-dependent fashion, but the effects were more profound in the OCI AML3 cells compared to the OCI AML2 cells (FIG. 14A and FIG. 18). To fully analyze the time-dependent impact of DOT1L inhibitor treatment on DNMT3A mutant AML cells, proliferation assays were performed for 14 days treating the DNMT3A-mutant cell lines OCI AML2 and OCI AML3 with 3 μM EPZ004777 or vehicle control. DOT1L inhibitor-sensitive MLLr cell line, MV411 was also included as a positive control and the MLL- and DNMT3A-wild-type cell line, HL60 as a negative control. There was no impact on the growth of the HL60 cells. The proliferation of the R882 DNMT3A-mutant OCI AML3 cells was profoundly inhibited beginning around 7 days of treatment, comparable to the growth kinetics of the MLLr cell line, MV411. Conversely, the growth of the non-R882 mutant OCI AML2 cells was only modestly slowed at this dose (FIG. 14B). The difference in responses between the R882 and non-R882 mutant cell lines likely reflects underlying biological differences between these two distinct mutations. Data from primary patient samples suggesting that the R882-mutant DNMT3A more profoundly impacts DNA methylation compared to non-R882 mutant DNMT3A, supports the idea that functional differences likely exist between R882- and non-R882 mutant DNMT3A.

Treatment with Pharmacologic Inhibitors of DOT1L Inhibits the Cellular Induces Apoptosis, Cell Cycle Arrest and Terminal Differentiation of DNMT3A Mutant AML Cell Lines

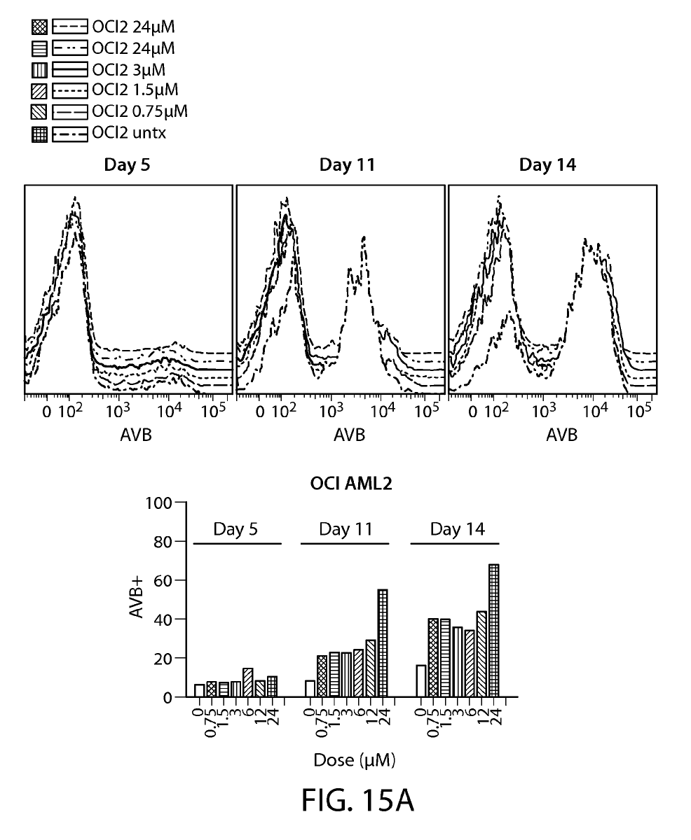

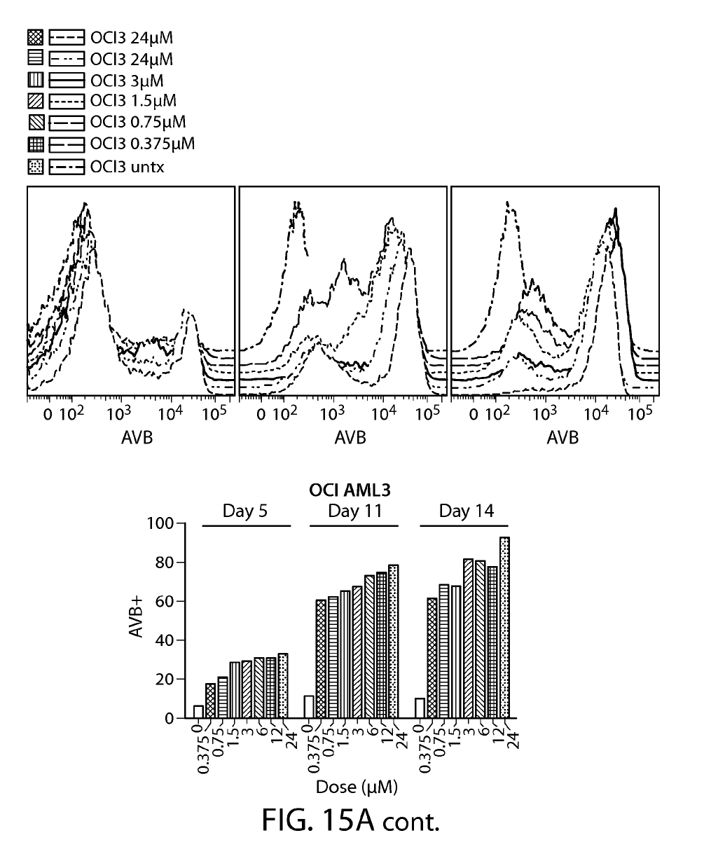

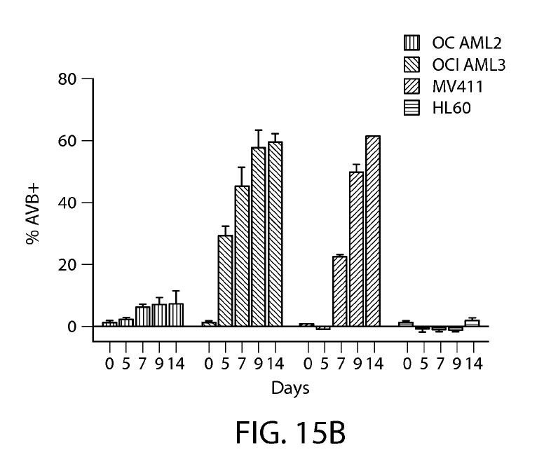

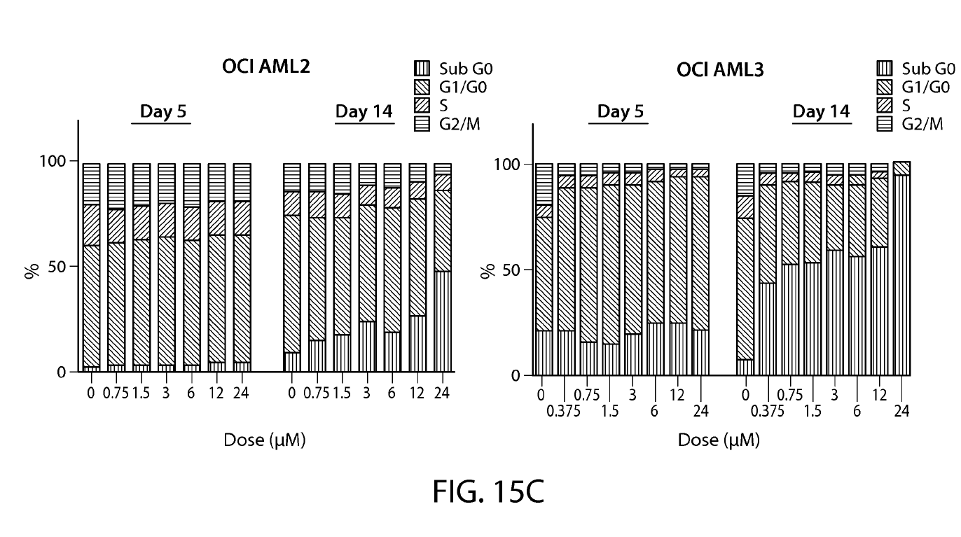

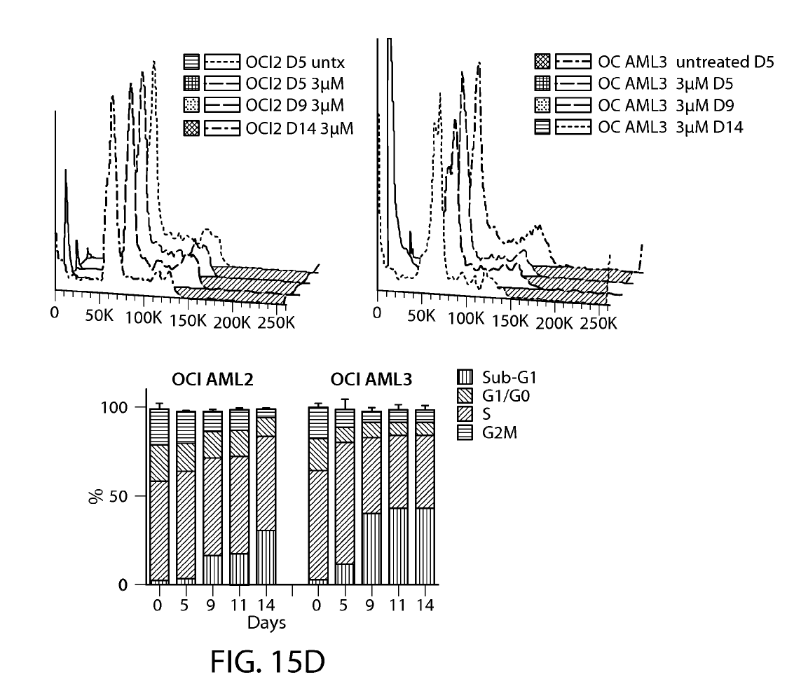

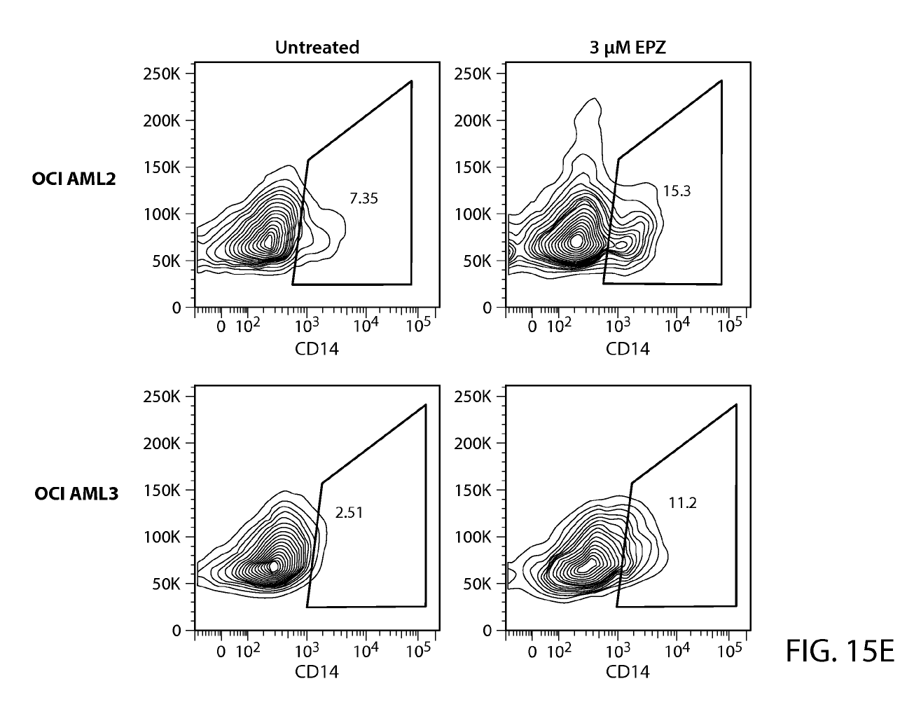

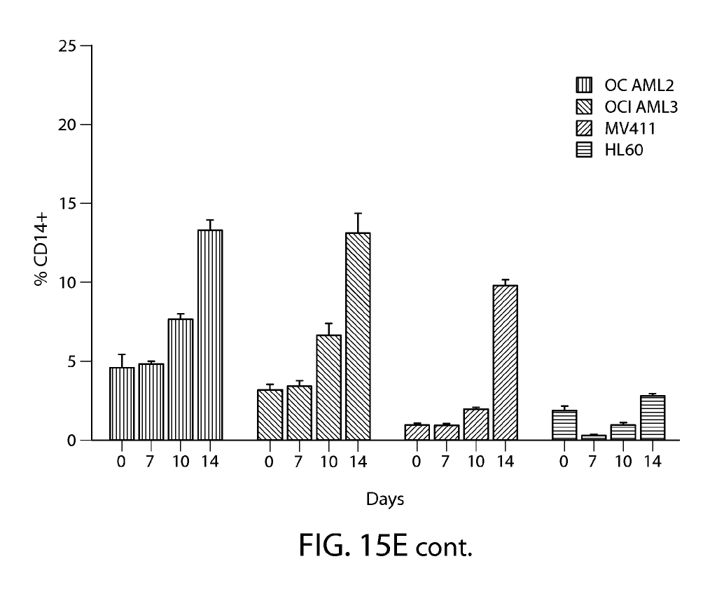

The mechanism of DOT1L inhibitor-induced cytotoxicity in DNMT3A mutant AML cells was investigated. By Annexin-V binding (AVB) flow cytometry assay, treatment with DOT1L inhibition led to a dose-dependent induction of apoptosis in both OCI AML2 and OCI AML3 cells, though higher doses were required in the OCI AML2 to achieve significant cellular death (FIG. 15A). Treatment with 3 μM EPZ004777 led to substantial induction of apoptosis in the OCI AML3 and MV411 cells in a time-dependent fashion beginning at 5-7 days of treatment (FIG. 15B). The HL60 cells had no induction of apoptosis and the OCI AML2 cells experienced minimal induction of apoptosis that plateaued at 7 days of therapy (FIG. 15B). The impact of DOT1L inhibitor treatment on cell cycle progression was also examined by flow cytometry for DNA content. Both OCI AML2 and OCI AML3 cells experienced cell cycle arrest with increased percentages of cells in sub-G1 and decreased percentages in S and G2/M phases in a dose- and time-dependent fashion (FIG. 15C and FIG. 15D). Both DNMT3A-mutant cell lines, OCI AML2 and OCI AML3 had evidence of induced differentiation with increased expression of the mature monocyte marker CD14, equivalent to that seen in the MV411 cells (FIG. 15E).

In vivo Efficacy of DOT1L Inhibition in a Nude Rat Xenograft

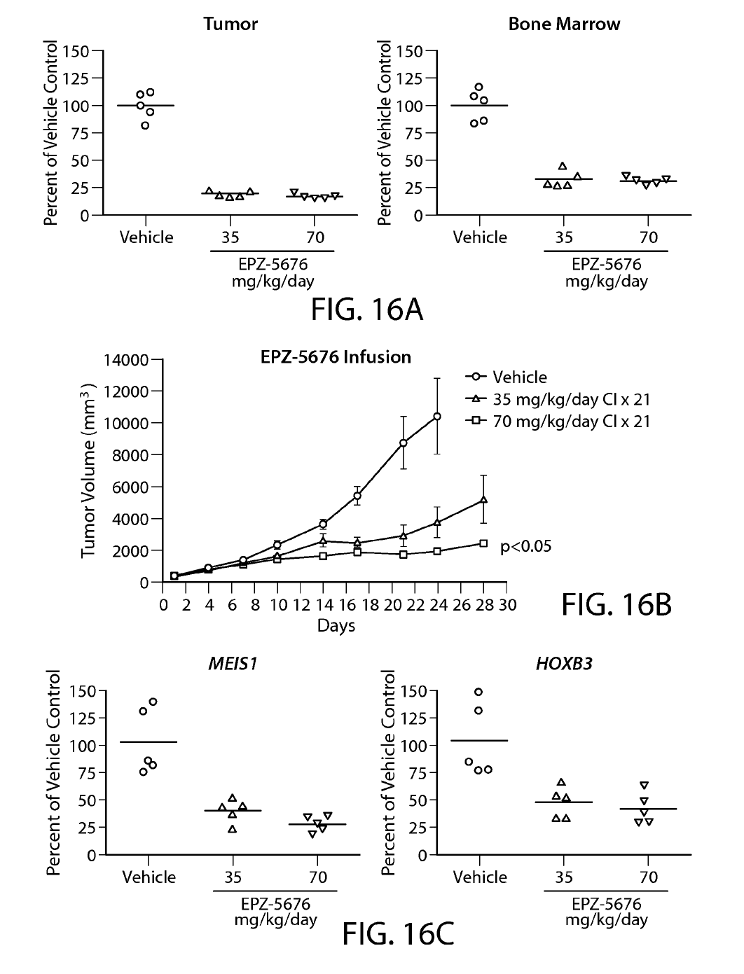

To test the in vivo efficacy of DOT1L inhibition on human DNMT3A-mutant AML, a nude rat xenograft model in which OCI AML3 cells were injected subcutaneously forming a leukemic tumor was utilized. After tumor engraftment, the rats (n=8 per treatment cohort) were treated with continuous intravenous infusion of vehicle control or DOT1L inhibitor compound, EPZ-5676, at 35 mg/kg/day or 70 mg/kg/day for 21 days. Tumor volume was measured daily until 28 days after the start of treatment or until the animal became moribund. The animals were then sacrificed and the tumors were harvested for measurement of H3K79me and gene expression. Both doses of EPZ-5676 led to significant reduction of H3K79me2 (FIG. 16A), resulting in repression of oncogenic genes including MEIS 1 and HOXB3, consistent with in vitro results (FIG. 16B). Tumor growth was inhibited in a dose dependent fashion reaching statistical significance in the 70 mg/kg/day cohort (FIG. 16C). These results indicate that DOT1L inihibition is effective in vivo for the treatment of DNMT3A-mutant AML.

DOT1L Inhibitor Treatment Selectively Inhibits the Colony-Forming Capacity and Induces Differentiation of Primary Patient Samples with DNMT3A Mutations.

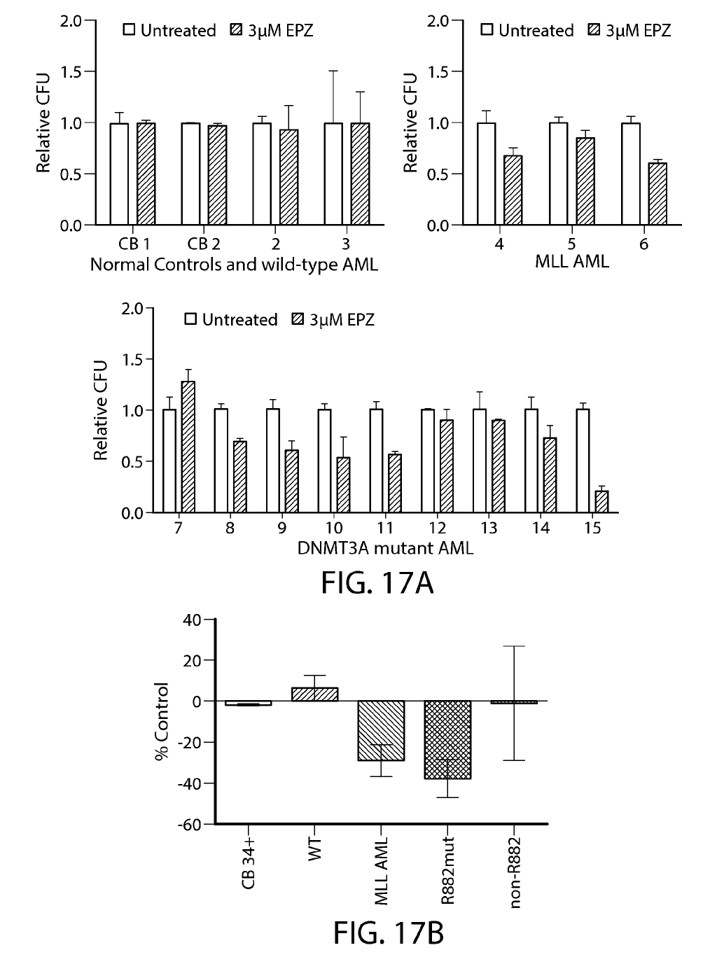

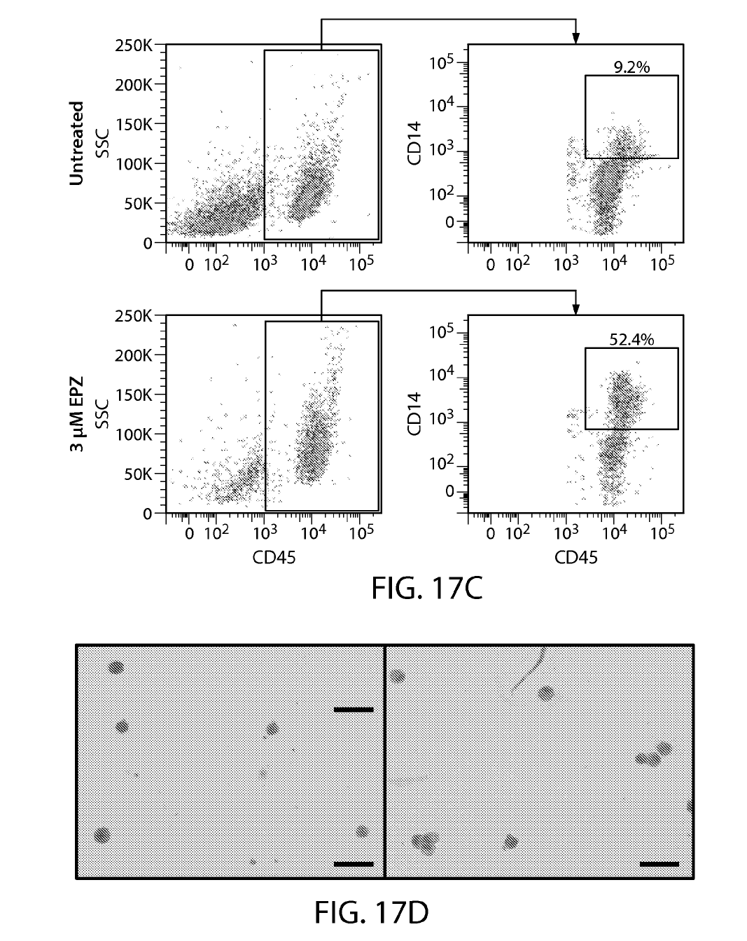

Effects of DOT1L inhibition in primary patient samples were also examined. Methylcellulose media supplemented with growth factors, which can support the growth of primary leukemia samples for several days, was used to study primary samples. After thawing the viably frozen human primary samples (sample characteristics are provided in Table 1), 5,000 viable cells/plate were cultured in human H4034 methylcellulose media (StemCell Technologies) plus 3 μM EPZ00477 or vehicle control. Treatment with 3 μM EPZ004777 did not significantly impact the colony-forming capacity (CFC) of normal CD34+ cells isolated from cord blood or primary AML patient samples with wild-type DNMT3A and MLL (FIG. 17A and FIG. 17B). EPZ004777 treatment caused reduced the colony-forming capacity of most samples with DNMT3A mutations comparable to the reduction seen in samples with MLL alterations with a possible increased sensitivity in samples with R882 DNMT3A mutations compared to non-R882 mutations (FIG. 17A and FIG. 17B).

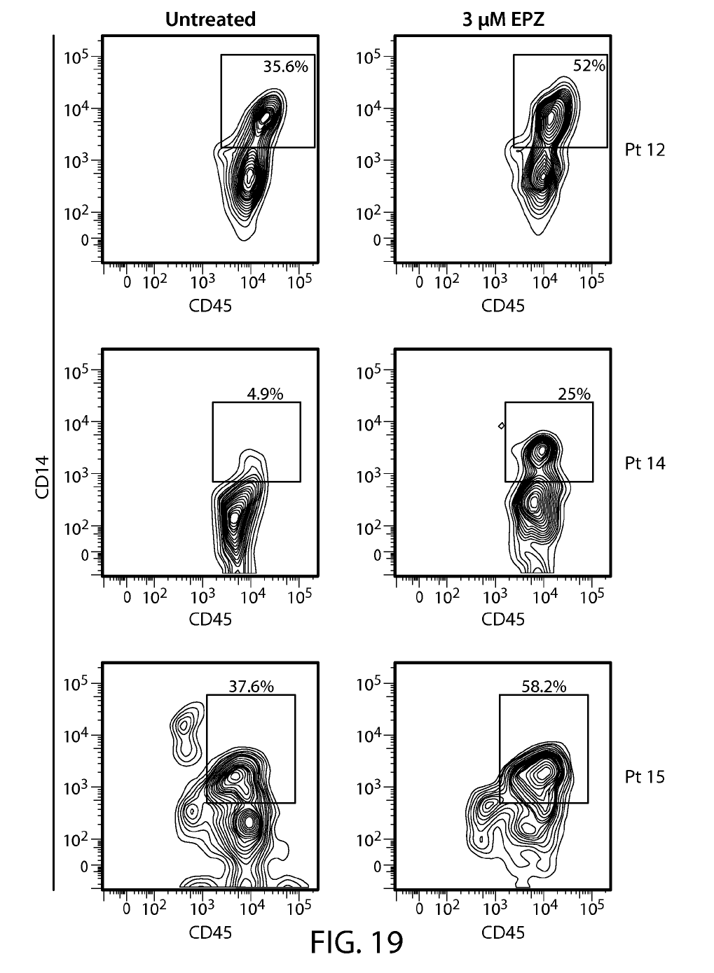

Cells from the plates were then isolated and flow cytometry was performed to identify evidence of differentiation. Treated cells demonstrated increased expression of the mature monocyte marker CD14 compared to controls (FIG. 17C and FIG. 19). Histologic evaluation of the isolated cells showed evidence of maturation with reduced nuclear to cytoplasmic ratio, increased granules in the cytoplasm and condensation of the nuclei (FIG. 17D). These results indicate that DOT1L inhibition reduces cellular proliferation and promotes differentiation of primary AML patient samples with DNMT3A mutations.

TABLE 1
Characteristics of cryopreserved AML primary patient samples and
results of colony forming assay.

| | | | | | | % |
|---|---|---|---|---|---|---|
| Sample ID | DNMT3A | MLL | NPM | FLT3 | Other | change |
| Patient 1* | WT | WT | WT | WT | -7/7q- | +157% |
| Patient 2 | WT | WT | WT | WT | -5/5q- | −6.7% |
| Patient 3 | WT | WT | n/d | D835 | Inv (16) | 0% |
| Patient 4 | n/d | MLLr | n/d | n/d | | −33.3% |
| | | (6; 11) | | | | |
| Patient 5 | n/d | MLLr | n/d | n/d | | −15.2% |
| | | (10; 11) | | | | |
| Patient 6 | WT | MLL Amp | WT | WT | | −40% |
| Patient 7 | N879D | WT | WT | WT | -7/7q- | +27% |
| Patient 8 | R882H | WT | WT | WT | TET2, | −30.7% |
| | | | | | KRAS | |
| Patient 9 | R882H | WT | WT | WT | | −39.6% |
| Patient 10 | R882S | WT | n/d | n/d | | −47.1% |
| Patient 11 | R882H | WT | WT | WT | -7/7q- | −44.1% |
| Patient 12 | R882H | WT | NPMc+ | ITD | | −11.1% |
| Patient 13 | R882H | WT | WT | WT | | −11.1% |
| Patient 14 | N8789D | WT | n/d | n/d | | −28.2% |
| Patient 15 | R882H | WT | n/d | n/d | | −79.5% |

Characteristics of cryopreserved AML primary patient samples and results of colony forming assay. Column one lists patient numbers of the primary AML patient samples treated in the colony forming assay (with corresponding patient numbers shown in Fig. 17 and Fig. 19). Columns 2-6 list the known molecular features of each sample. Column 7 lists the percent change in colony forming capacity of EPZ004777-treated (3 μm) relative to vehicle control-treated sample, calculated using the formula: 100 − ((average colonies per EPZ-treated plate/average colonies per vehicle control-treated plate) × 100).
*sample not included in Fig. 7A,
n/d, not determined;
WT, wild-type;
mut, mutation at unspecified site;
MLLr, MLL rearranged;
MLL amp, MLL amplified;
ITD, internal tandem duplication mutation.

1. A method of treating acute myeloid leukemia (AML), the method comprising administering a composition comprising a DOT1L inhibitor to a subject having AML associated with one or more DNMT3A mutations.

2. The method of claim 1, wherein the one or more DNMT3A mutations cause a dominant negative loss of function of DNMT3A activity.

3. The method of claim 1, wherein a biological sample from the subject is identified as having one or more expanded DNA methylation canyons.

4. The method of claim 3, wherein the one or more expanded DNA methylation canyons comprise a HOX gene cluster.

5. The method of claim 4, wherein the HOX gene cluster is a HOXA gene cluster.

6. The method of claim 4, wherein the HOX gene cluster is a HOXB gene cluster.

7. The method of claim 1, wherein a biological sample from the subject is identified as having a level of H3K79me2 that is higher than in a DNMT3A wild-type control.

8. A method of treating a subject having AML, the method comprising:
a. obtaining a biological sample from the subject;
b. detecting the presence of one or more DNMT3A mutations in the biological sample, detecting the presence of one or more expanded DNA methylation canyons in the biological sample, or detecting the presence of a higher level of H3K79me2 in the biological sample than in a DNMT3A wild-type control; and,
c. administering to the subject a composition comprising a DOT1L inhibitor.

9.-10. (canceled)

11. A method of identifying a subject having AML that is responsive to treatment with a DOT1L inhibitor, the method comprising:
a. obtaining a biological sample from the subject;
b. assaying the biological sample for the presence of one or more DNMT3A mutations, assaying the biological sample for the presence of one or more expanded DNA methylation canyons, or assaying a level of H3K79me2 in the biological sample; and,
c. identifying the subject as responsive to treatment with a DOT1L inhibitor if one or more DNMT3A mutations are detected in the biological sample, if one or more expanded DNA methylation canyons are detected in the biological sample, or if the level of H3K79me2 is higher than in a DNMT3A wild-type control.

12.-13. (canceled)

14. The method of claim 1, the DOT1L inhibitor is a compound of formula: 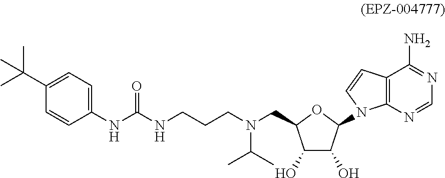 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]
or a pharmaceutically acceptable salt thereof.

15. The method of claim 1, wherein the DOT1L inhibitor is a compound of formula: 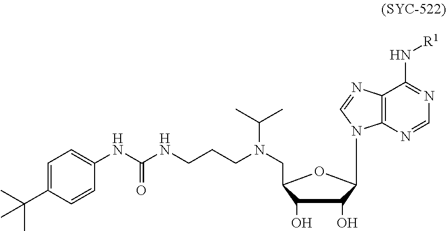
(SYC-522)
wherein R1 is a H, or a pharmaceutically acceptable salt thereof.

16. The method of claim 1, wherein the DOT1L inhibitor is a compound of formula: 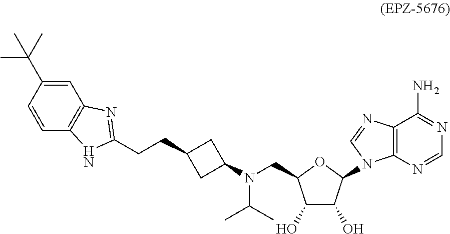 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]
or a pharmaceutically acceptable salt thereof.

17. The method of claim 8, wherein the biological sample is selected from the group consisting of bone marrow, peripheral blood cells, blood, cerebrospinal fluid, skin lesions, chloroma biopsy, plasma, serum, urine, saliva and a cell.

18. The method of claim 1, wherein the presence of one or more mutations of DNMT3A are detected by genome sequence analysis, next generation sequencing, and/or PCR-based mutation detection.
